SEQUENCE LISTING (38 sequences)

SEQ ID NO: 1 - protein, 8 aa, Artificial
  Synthetic
LAAAVSNF

SEQ ID NO: 2 - protein, 8 aa, Artificial
  Synthetic
QPAHLTFP

SEQ ID NO: 3 - protein, 8 aa, Artificial
  Synthetic
  MISC_FEATURE (1)..(1): “Xaa” may be any amino acid
  MISC_FEATURE (4)..(4): “Xaa” may be “Ser” or “Thr”
  MISC_FEATURE (5)..(5): “Xaa” may be a hydrophobic amino acid, i.e., “Ala,” “Cys,” “Ile,” “Leu,” “Met,” “Phe,” “Pro,” “Trp,” “Tyr” or “Val”
  MISC_FEATURE (6)..(6): “Xaa” may be “Ser” or “Thr”
  MISC_FEATURE (7)..(8): “Xaa” may be any amino acid
XPRXXXXX

SEQ ID NO: 4 - protein, 8 aa, Artificial
  Synthetic
  MISC_FEATURE (1)..(1): “Xaa” may be any amino acid
  MISC_FEATURE (2)..(2): “Xaa may be ”Ala,“ ”Gly“ or ”Leu“
  MISC_FEATURE (3)..(3): ”Xaa“ may be ”Arg,“ ”Leu“ or ”Ala“
  MISC_FEATURE (4)..(4): ”Xaa“ may be ”Glu,“ ”Lus,“ ”His,“ ”Ala“ or ”Arg“
  MISC_FEATURE (5)..(5): ”Xaa“ may be a hydrophobic amino acid, i.e., ”Ala,“ ”Cys,“ ”Ile,“ ”Leu,“ ”Met,“ ”Phe,“ ”Pro,“ ”Trp,“ ”Tyr“ or ”Val“
  MISC_FEATURE (6)..(6): ”Xaa“ may be ”Leu,“ ”Lys,“ ”Gly“ or ”Ala“
  MISC_FEATURE (7)..(7): ”Xaa“ may be ”Gly,“ ”Asp,“ ”Ala“ or ”Asn“
  MISC_FEATURE (8)..(8): ”Xaa“ may be any amino acid
XXXXXXXX

SEQ ID NO: 5 - protein, 8 aa, Artificial
  Synthetic
GAEQRTES

SEQ ID NO: 6 - protein, 8 aa, Artificial
  Synthetic
PDIHGTYK

SEQ ID NO: 7 - protein, 8 aa, Artificial
  Synthetic
FDSRKTSL

SEQ ID NO: 8 - protein, 8 aa, Artificial
  Synthetic
LDSDLSCK

SEQ ID NO: 9 - protein, 8 aa, Artificial
  Synthetic
LDEERTVR

SEQ ID NO: 10 - protein, 8 aa, Artificial
  Synthetic
KELLDTVT

SEQ ID NO: 11 - protein, 8 aa, Artificial
  Synthetic
GEVTKSLQ

SEQ ID NO: 12 - protein, 8 aa, Artificial
  Synthetic
IEESLTSE

SEQ ID NO: 13 - protein, 8 aa, Artificial
  Synthetic
EESLTSEF

SEQ ID NO: 14 - protein, 8 aa, Artificial
  Synthetic
AGTTPSPG

SEQ ID NO: 15 - protein, 8 aa, Artificial
  Synthetic
KGQWVTKF

SEQ ID NO: 16 - protein, 8 aa, Artificial
  Synthetic
EKSYGTRP

SEQ ID NO: 17 - protein, 8 aa, Artificial
  Synthetic
PKLKLSYE

SEQ ID NO: 18 - protein, 8 aa, Artificial
  Synthetic
GKLARSTK

SEQ ID NO: 19 - protein, 8 aa, Artificial
  Synthetic
TLIEESLT

SEQ ID NO: 20 - protein, 8 aa, Artificial
  Synthetic
KLRIKSSF

SEQ ID NO: 21 - protein, 8 aa, Artificial
  Synthetic
KLARSTKE

SEQ ID NO: 22 - protein, 8 aa, Artificial
  Synthetic
LLDTVTAP

SEQ ID NO: 23 - protein, 8 aa, Artificial
  Synthetic
LPLTGSMS

SEQ ID NO: 24 - protein, 8 aa, Artificial
  Synthetic
SPDIHGTY

SEQ ID NO: 25 - protein, 8 aa, Artificial
  Synthetic
IPDEISIL

SEQ ID NO: 26 - protein, 8 aa, Artificial
  Synthetic
APLEKSYG

SEQ ID NO: 27 - protein, 8 aa, Artificial
  Synthetic
RPRVLTGN

SEQ ID NO: 28 - protein, 8 aa, Artificial
  Synthetic
KPIKLTQV

SEQ ID NO: 29 - protein, 8 aa, Artificial
  Synthetic
SPDFSKIT

SEQ ID NO: 30 - protein, 8 aa, Artificial
  Synthetic
SPLSVATA

SEQ ID NO: 31 - protein, 8 aa, Artificial
  Synthetic
DPKAVLRY

SEQ ID NO: 32 - protein, 8 aa, Artificial
  Synthetic
LPLKVTQN

SEQ ID NO: 33 - protein, 8 aa, Artificial
  Synthetic
EQRTESII

SEQ ID NO: 34 - protein, 8 aa, Artificial
  Synthetic
QSLFDSPD

SEQ ID NO: 35 - protein, 8 aa, Artificial
  Synthetic
DSRKTSLE

SEQ ID NO: 36 - protein, 8 aa, Artificial
  Synthetic
VTKFDSRK

SEQ ID NO: 37 - protein, 8 aa, Artificial
  Synthetic
YYDLISSP

SEQ ID NO: 38 - protein, 8 aa, Artificial
  Synthetic
LYRVRSSM

(19) United States
(12) Patent Application Publication
(10) Pub. No.: US 2007/0232541 A1
(43) Pub. Date: Oct. 4, 2007

(54) METHODS OF REGULATING ANGIOGENISIS THROUGH STABILIZATION OF PEDF

(75) Inventors: Christopher Reiter, Wheaton, MD (US); Miguel E. Carrion, New Market, MD (US); Lisa Wei, Gaithersburg, MD (US); Melissa M. Hamilton, Gaithersburg, MD (US); C. Richter King, Washington, DC (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: GenVec, Inc., Gaithersburg, MD (US)

(21) Appl. No.: 11/750,510

(22) Filed: May 18, 2007

Related U.S. Application Data
(63) Continuation of application No. PCT/US05/42266, filed on Nov. 18, 2005.
(60) Provisional application No. 60/629,372, filed on Nov. 19, 2004.

(51) Int. Cl. A61K 38/16; C12N 15/63

(52) U.S. Cl. 514/12; 435/455

(57) ABSTRACT

The invention provides methods of inhibiting angiogenesis within a tissue, promoting neuron protection within a tissue, and treating a disease in a mammal, wherein (a) a gene transfer vector encoding a protein of a serpin superfamily or a therapeutic fragment or variant thereof or (b) a protein of a serpin superfamily or a therapeutic fragment or variant thereof is administered with an inhibitor of matrix metalloprotease (MMP). Alternatively, the method comprises administration of (a) a protein of a serpin superfamily or therapeutic fragment or variant thereof or (b) a gene transfer vector encoding a protein of a serpin superfamily or a therapeutic fragment or variant thereof, wherein the protein or nucleic acid encoding the protein comprises at least one mutation which renders the protein of the serpin superfamily resistant to cleavage by an MMP.

### CROSS-REFERENCE TO RELATED APPLICATIONS

This patent application is continuation of copending International Patent Application No. PCT/US2005/042266, filed Nov. 18, 2005, which claims the benefit of U.S. Provisional Patent Application No. 60/629,372, filed Nov. 19, 2004.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 8,030 Byte ASCII (Text) file named “701595_ST25.TXT,” created on May 18, 2007.

### BACKGROUND OF THE INVENTION

Angiogenesis is a rapidly activated, yet tightly controlled, process that leads to the development of new capillaries and blood vessels. The process is induced and sustained by a number of protein factors such as VEGF, PDGF, IL-8, FGF-2, as well as others playing a major role. The process is inhibited by the protein, PEDF. New vessels are formed as a function of an altered balance between the regional levels of pro-angiogenic proteins (VEGF, FGF, etc.) and anti-angiogenic proteins (PEDF, endostatin, angiostatin, etc).

The balance between angiogenesis and anti-angiogenesis is predominantly in the direction of zero vessel growth. This is reflected in the presence of nearly micromolar levels of PEDF in human serum and in intravitreal fluid, which constitutively inhibits aberrant vessel growth. When angiogenesis is required, the balance must swing away from anti-angiogenesis towards a more angiogenic profile. An angiogenic stimulus, such as regional hypoxia, rapidly induces VEGF expression and initiates the shift to promote angiogenesis. In the presence of higher levels of VEGF, secondary proteins are expressed that prepare tissue for new vessels. One set of proteins induced by the presence of VEGF is the matrix metalloproteases (MMPs), a family of enzymes that maintain and remodel tissue architecture.

In some diseases, such as wet age-related macular degeneration and diabetic retinopathy, levels of PEDF decrease in patients and aberrant cell growth is observed. Thus, there remains a need for an effective inhibitor of angiogenesis for the prophylactic and therapeutic treatment of diseases (e.g., tumor growth, arthritis, wet age-related macular degeneration, diabetic retinopathy, proliferative diabetic retinopathy, cataract formation, and uveitis, amyotrophic lateral sclerosis (Lou Gehrig's Disease), and stroke). The present invention provides materials and methods for inhibiting angiogenesis, promoting neuron protection or enhanced neuronal function, and treating diseases related to the inhibition of angiogenesis. These and other advantages of the present invention will become apparent from the detailed description provided herein.

### BRIEF SUMMARY OF THE INVENTION

The invention provides a method of inhibiting angiogenesis within a tissue, which method comprises contacting the tissue with (a) a gene transfer vector comprising a nucleic acid sequence encoding a protein of a serpin superfamily or therapeutic fragment or variant thereof and (b) an inhibitor of a matrix metalloprotease (MMP), wherein the nucleic acid sequence encoding the protein of a serpin superfamily or therapeutic fragment or variant thereof is expressed, and angiogenesis within the tissue is inhibited.

The invention provides a method of promoting neuron protection within a tissue, which method comprises contacting the tissue with (a) a gene transfer vector comprising a nucleic acid sequence encoding a protein of a serpin superfamily or therapeutic fragment or variant thereof and (b) an inhibitor of a matrix metalloprotease (MMP), wherein the nucleic acid sequence encoding the protein of a serpin superfamily or therapeutic fragment or variant thereof is expressed, and neuron protection within the tissue is promoted.

The invention provides a method of inhibiting angiogenesis within a tissue, which method comprises contacting the tissue with a gene transfer vector comprising a nucleic acid sequence encoding a protein of a serpin superfamily or therapeutic fragment or variant thereof, wherein the nucleic acid sequence comprises at least one mutation which renders the protein of the serpin superfamily resistant to cleavage by a matrix metalloprotease (MMP), and wherein the nucleic acid sequence encoding the protein of the serpin superfamily or therapeutic fragment or variant thereof is expressed, and angiogenesis within the tissue is inhibited.

The invention encompasses a method of promoting neuron protection within a tissue, which method comprises contacting the tissue with a gene transfer vector comprising a nucleic acid sequence encoding a protein of a serpin superfamily or therapeutic fragment or variant thereof, wherein the nucleic acid sequence comprises at least one mutation which renders the protein of the serpin superfamily resistant to cleavage by a matrix metalloprotease (MMP), and wherein the nucleic acid sequence encoding the protein of the serpin superfamily is expressed or therapeutic fragment or variant thereof, and neuron protection within the tissue is promoted.

The invention also encompasses a method of inhibiting angiogenesis within a tissue, which method comprises contacting the tissue with (a) a protein of a serpin superfamily or therapeutic fragment or variant thereof and (b) an inhibitor of a matrix metalloprotease (MMP), wherein angiogenesis within the tissue is inhibited.

The invention provides a method of promoting neuron protection within a tissue, which method comprises contacting the tissue with (a) a protein of a serpin superfamily or therapeutic fragment or variant thereof and (b) an inhibitor of a matrix metalloprotease (MMP), wherein the neuron protection within the tissue is promoted.

The invention further provides a method of inhibiting angiogenesis within a tissue, which method comprises contacting the tissue with a protein of a serpin superfamily or a therapeutic fragment or variant thereof, wherein the protein of the serpin superfamily is resistant to cleavage by a matrix metalloprotease (MMP), and wherein angiogenesis within the tissue is inhibited.

Also provided by the invention is a method of promoting neuron protection within a tissue, which method comprises contacting the tissue with a protein of a serpin superfamily or a therapeutic fragment or variant thereof, wherein the protein of the serpin superfamily is resistant to cleavage by a matrix metalloprotease (MMP), and wherein neuron protection within the tissue is promoted.

The invention provides a method of treating a disease in a mammal, which method comprises administering to the mammal (a) a gene transfer vector comprising a nucleic acid sequence encoding protein of a serpin superfamily or therapeutic fragment or variant thereof and (b) an inhibitor of a matrix metalloprotease (MMP), wherein the nucleic acid sequence encoding the protein of a serpin superfamily or therapeutic fragment or variant thereof is expressed, thereby treating the disease in the mammal.

The invention provides a method of treating a disease in a mammal, which method comprises administering to the mammal a gene transfer vector comprising a nucleic acid sequence encoding a protein of a serpin superfamily or therapeutic fragment or variant thereof, wherein the nucleic acid sequence comprises at least one mutation which renders the protein of the serpin superfamily or therapeutic fragment or variant thereof resistant to cleavage by a matrix metalloprotease (MMP), and wherein the nucleic acid sequence encoding the protein of the serpin superfamily or therapeutic fragment or variant thereof is expressed, thereby treating the disease in the mammal.

Additionally, the invention provides a method of treating a disease in a mammal, which method comprises administering to the mammal (a) a protein of a serpin superfamily or therapeutic fragment or variant thereof and (b) an inhibitor of a matrix metalloprotease (MMP), thereby treating the disease in the mammal.

The invention encompasses a method of inhibiting vascular permeability in a tissue, which method comprises administering to the mammal (a) a gene transfer vector comprising a nucleic acid sequence encoding protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed, thereby inhibiting vascular permeability in the tissue.

The invention also encompasses a method of inhibiting vascular permeability in a tissue, which method comprises administering to the mammal (a) a protein of a serpin superfamily or a therapeutic fragment or variant thereof and (b) an inhibitor of a matrix metalloprotease (MMP), thereby inhibiting vascular permeability in the tissue.

### BRIEF DESCRIPTION OF THE DRAWINGS

FIG. 1 demonstrates dose dependent inhibition of endothelial cell growth by the administration of PEDF at various concentrations. Images were captured 5 days after preparing the mouse aortic ring assay. +GF refers to 25 ng/mL of FGF and 2.5 ng/mL of VEGF.

FIG. 2 is a graph indicating the endothelial cell count 3 days (72 hours) after preparing the mouse aortic ring assay.

FIG. 3 is a graph indicating the endothelial cell count 3 days (72 hours) after preparing the mouse aortic ring assay at 1, 10, and 100 nM concentrations of PEDF with and without the presence of MMP-9.

FIG. 4 is an SDS-PAGE demonstrating PEDF cleavage products produced by treatment with MMP-9 and MMP-2.

FIG. 5 is an SDS-PAGE demonstrating PEDF cleavage products produced by treatment with MMP-9, MMP-2, and MMP-7.

FIG. 6 is a schematic depiction of a method to identify MMP-9 cleavage sites of a protein susceptible to MMP-9 cleavage.

FIG. 7 is a schematic depiction of the known and proposed cleavage sites of MMP-9.

FIG. 8A is a graph of a MALDI mass spectrum of a peptide from a peak in the HPLC chromatograph of the MMP-9-treated PEDF sample after tryptic digest. FIG. 8B is a graph of a MALDI mass spectrum of peptides isolated from the MMP-9-treated (lower trace) and untreated PEDF (upper trace) after tryptic digest.

FIG. 9 is a graph indicating pg of PEDF protein per total μg of protein.

FIG. 10 is a graph indicating pg of PEDF protein per total μg of protein in mouse eyes treated with AdPEDF alone or in combination with SB-3CT. Data is represented with box (25-50-75 quartile) and whiskers (5-95 quartile) with overlaid data from each animal.

FIG. 11 is a graph demonstrating endothelial cell invasion induced in primary dermal microvascular endothelial cells treated with PEDF or PEDF in combination with MMP-9.

### DETAILED DESCRIPTION OF THE INVENTION

The invention is predicated on the discovery that certain matrix metalloproteases (MMPs), such as MMP-2 and MMP-9, cleave members of the serpin superfamily of proteins, such as pigment epithelium-derived factor (PEDF). This discovery has multiple implications in the treatment of diseases, such as diseases related to angiogenesis and the protection of neurons.

Specifically, this discovery indicates that inhibitors of MMPs are useful in improving the activity of a serpin protein (e.g., as administered by a gene transfer vector). The inhibition of MMP provides for a stabilized serpin protein (e.g., PEDF) and, thus, longer duration and higher activity of the serpin protein (e.g., PEDF).

Accordingly, the invention is directed to a method of inhibiting angiogenesis within a tissue, which method comprises contacting the tissue with (a) a gene transfer vector comprising a nucleic acid sequence encoding a protein of a serpin superfamily or a therapeutic fragment or variant thereof and (b) an inhibitor of a matrix metalloprotease (MMP), wherein the nucleic acid sequence encoding the protein of a serpin superfamily or therapeutic fragment or variant thereof is expressed, and angiogenesis within the tissue is inhibited.

Similarly, the invention is directed to a method of inhibiting angiogenesis within a tissue, which method comprises contacting the tissue with (a) a protein of a serpin superfamily or therapeutic fragment or variant thereof and (b) an inhibitor of an MMP, wherein angiogenesis within the tissue is inhibited.

The invention also encompasses a method of promoting neuron protection within a tissue, which method comprises contacting the tissue with (a) a gene transfer vector comprising a nucleic acid sequence encoding a protein of a serpin superfamily or therapeutic fragment or variant thereof and (b) an inhibitor of an MMP, wherein the nucleic acid sequence encoding the protein of a serpin superfamily or therapeutic fragment or variant thereof is expressed, and neuron protection within the tissue is promoted.

Similarly, the invention is directed to a method of promoting neuron protection within a tissue, which method comprises contacting the tissue with (a) a protein of a serpin superfamily or therapeutic fragment or variant thereof and (b) an inhibitor of an MMP, wherein neuron protection is promoted.

The invention also is directed to a method of inhibiting vascular permeability in a tissue, which method comprises administering to the mammal (a) a gene transfer vector comprising a nucleic acid sequence encoding protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed, thereby inhibiting vascular permeability in the tissue.

Similarly, the invention is directed to a method of inhibiting vascular permeability (e.g., VEGF-induced vascular permeability) in a tissue, which method comprises administering to the mammal (a) a protein of a serpin superfamily or a therapeutic fragment or variant thereof and (b) an inhibitor of a matrix metalloprotease (MMP), thereby inhibiting vascular permeability in the tissue.

Serine proteinase inhibitors (serpins) are a group of structurally related proteins, which share many of the same characteristics, including ranging from about 400 to 500 amino acids in length, being an extracellular protein, and functioning as irreversible inhibitors with the well-defined structural-functional characteristic of a reactive region that acts as “bait” for an appropriate serine protease. This reactive region is found in the C-terminal of the serpin proteins. The proteins of the serpin superfamily usually share at least an 11 amino acid pattern, wherein:

The amino acid at position 1 can be leucine, isoleucine, valine, methionine, phenylalanine, or tyrosine. Most likely, the amino acid in the first position is a tyrosine.

The amino acid at position 2 can be any amino acid.

The amino acid at position 3 can be leucine, isoleucine, valine, methionine, phenylalanine, tyrosine, alanine, or cysteine. Most likely, the amino acid in the third position is a leucine.

The amino acid at position 4 can be aspartate, asparagine, or glutamine. Most likely, the amino acid in the fourth position is asparagine.

The amino acid at position 5 can be arginine, lysine, histidine, glutamine, and serine. Most likely, the amino acid in the fifth position glutamine.

The amino acid at position 6 can be proline, serine, or threonine. Most likely, the amino acid in the sixth position is proline.

The amino acid at position 7 is phenylalanine.

The amino acid at position 8 can be leucine, isoleucine, valine, methionine, phenylalanine, or tyrosine. Most likely, the amino acid in the eighth position is isoleucine.

The amino acid at position 9 can be leucine, isoleucine, valine, methionine, phenylalanine, tyrosine, or cysteine. Most likely, the amino acid in the ninth position is a phenylalanine.

The amino acid at position 10 can be any amino acid.

The amino acid at position 11 can be leucine, isoleucine, valine, methionine, phenylalanine, alanine, or histidine. Most likely, the amino acid in the eleventh position is leucine.

Proteins of the serpin superfamily include alpha-1 protease inhibitor (alpha-1-antitrypsin, contrapsin), alpha-1-antichymotrypsin, antithrombin III, alpha-2-antiplasmin, heparin cofactor II, complement C1 inhibitor, plasminogen activator inhibitors 1 (PAI-1) and 2 (PAI-2), glia derived nexin (GDN) (protease nexin I), protein C inhibitor, rat hepatocytes SPI-1, SPI-2 and SPI-3 inhibitors, human squamous cell carcinoma antigen (SCCA) (which may act in the modulation of the host immune response against tumor cells), a lepidopteran protease inhibitor, leukocyte elastase inhibitor (which in contrast to other serpins is an intracellular protein), neuroserpin (a neuronal inhibitor of plasminogen activators and plasmin), cowpox virus crmA (an inhibitor of the thiol protease interleukin-1B converting enzyme (ICE), which is the only serpin known to inhibit a non-serine proteinase), and certain orthopoxvirus protease inhibitors (which may be involved in the regulation of the blood clotting cascade and/or of the complement cascade in the mammalian host). On the basis of strong sequence similarities, a number of proteins with no known inhibitory activity are said to belong to the serpin superfamily, including bird ovalbumin (and the related proteins X and Y), angiotensinogen (the precursor of the angiotensin active peptide), barley protein Z (the major endosperm albumin), corticosteroid binding globulin (CBG), thyroxine-binding globulin (TBG), sheep uterine milk protein (UTMP), pig uteroferrin-associated protein (UFAP), Hsp47 (an endoplasmic reticulum heat-shock protein that binds strongly to collagen and could act as a chaperone in the collagen biosynthetic pathway), maspin (which seems to function as a tumor suppressor), PEDF (a protein with a strong neutrophic activity), and Ep45 (an estrogen-regulated protein from Xenopus).

Preferably, the protein of the serpin superfamily is PEDF or a therapeutic fragment or variant thereof. PEDF is a 48 KDa member of the serpin superfamily of proteins and has been found to suppress the angiogenic activities of a number of growth factors such as VEGF, FGF-2, PDGF, and IL-8. PEDF also has been shown to inhibit vascular permeability (e.g., VEGF-induced vascular permeability) (see, e.g., Liu et al., PNAS, 101(17), 6605-6610 (2004)).

A functioning PEDF peptide or a therapeutic fragment or variant thereof prevents or ameliorates neovascularization. In that PEDF also has neurotrophic activity, a functioning PEDF peptide or a therapeutic fragment or variant thereof desirably promotes neuronal cell differentiation, inhibits glial cell proliferation, and/or promotes neuronal cell survival (i.e., promotes neuron protection).

One of ordinary skill in the art will understand that complete prevention or amelioration of neovascularization is not required in order to realize a therapeutic effect. Likewise, complete induction of neuron survival or differentiation is not required in order to realize a benefit. Therefore, both partial and complete prevention and amelioration of angiogenesis or promotion of neuron survival is appropriate. The ordinarily skilled artisan has the ability to determine whether a modified serpin protein or a fragment (e.g., PEDF or fragment thereof) has neurotrophic, anti-angiogenic, or anti-vasopermeability therapeutic activity using, for example, neuronal cell differentiation and survival assays (see, for example, U.S. Pat. No. 5,840,686), the mouse ear model of neovascularization, the rat hindlimb ischemia model, mouse aortic ring assay, and other assays known in the art.

A therapeutic fragment refers to those fragments (e.g., peptides) having biological activity sufficient to, for example, inhibit angiogenesis or promote neuron survival. A variant refers to nucleic acid sequences comprising substitutions, deletions, or additions, but which encode a functioning serpin protein (e.g., a PEDF peptide) or a therapeutic fragment thereof, or the corresponding functioning serpin protein or therapeutic fragment thereof. Additionally, a fusion protein comprising a serpin protein or a therapeutic fragment or variant thereof and, for example, a moiety that stabilizes peptide conformation, also are encompassed by the invention.

MMPs are enzymes that are able to degrade most components of the extracellular matrix, such as collagens, laminins, fibronectins, elastins, and the protein core of proteoglycans (see, e.g., Hoekstra et al., The Oncologist, 6, 415-427 (2001)). Members of the family include collagenases (MMP-1, MMP-8, MMP-13, and MMP-18), stromelysins (MMP-3, MMP-7, and MMP-10), gelatinases (MMP-2 and MMP-9), membrane type (MMP-14, MMP-15, MMP-16, MMP-17, and MMP-18), and others (MMP-11, MMP-12, MMP-19, MMP-20, MMP-23, and MMP-24) (see, e.g., Hoekstra et al., supra). Preferably, the MMP (e.g., to be inhibited) is selected from the group consisting of MMP-1, MMP-3, MMP-7, MMP-9, and MMP-12. Under normal physiological conditions, MMPs contribute to tissue remodeling during development, wound healing, and the menstrual cycle. Under pathological conditions, MMPs have been involved in diseases afflicted by the destruction of connective tissue such as rheumatoid arthritis and cancer. MMPs also play a regulatory role by processing matrix proteins, cytokines, growth factors, and adhesion molecules to generate fragments with enhanced or reduced biological effects.

Inhibitors of MMPs include, but are not limited to, tissue inhibitors of metalloproteases (TIMP), Batimastat, Marimastat, AG3340 (prinomastat), BAY 12-9566, MM1270, COL-3 (metastat), BMS-275291, CP-471,358, AE-941 (neovastat), SB-3CT (Cal Biochem), tetracycline and tetracycline derivatives (e.g., Periostat™), or other inhibitors of MMPs known in the art (see, e.g., Hoekstra et al., supra). Preferably, the inhibitor of MMPs is TIMP or a fragment or variant thereof, such as TIMP-1, TIMP-2, TIMP-3, and TIMP-4.

The tissue in which angiogenesis is inhibited and/or neuron protection is promoted can be any suitable tissue. Desirably, the tissue is a mammalian tissue, such as tissue from mice, rats, cats, dogs, guinea pigs, hamsters, rabbits, cats, dogs, pigs, cows, horses, primates, and humans. Preferably, the tissue comprises human cells, such as cells of neural origin, cells of the inner ear (e.g., hair cells), photoreceptor cells, bipolar cells, cerebellar granular cells, iris epithelial cells, interstitial cells, muscle cells, connective tissue cells, scleral cells, corneal cells, Mueller cells, ciliary epithelial cells, retinal pigment epithelial cells, glial cells, fibroblasts, endothelial cells, astrocytes, or cells of the trabecular meshwork.

The protein of the serpin superfamily (or the gene transfer vector comprising a nucleic acid sequence encoding the protein) and the inhibitor of an MMP can be administered in any suitable manner, either simultaneously or separately in either order, once or in multiple doses (e.g., two, three, four, five, six, seven, eight, nine, ten, or more doses). When the inhibitor of an MMP and the protein of serpin superfamily (or the gene transfer vector comprising a nucleic acid sequence encoding the protein) are administered separately, it is preferable to administer the inhibitor of an MMP before the protein of serpin superfamily (or the gene transfer vector comprising a nucleic acid sequence encoding the protein). For example, the inhibitor of an MMP can be administered at least about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, or about 48 hours before the protein of serpin superfamily (or the gene transfer vector comprising a nucleic acid sequence encoding the protein). Alternatively, the inhibitor of an MMP inhibitor can be administered continuously over a particular time period before, during, or following administration of the protein of serpin superfamily (or the gene transfer vector comprising a nucleic acid sequence encoding the protein), such as an oral dosage form with continuous or time-elapsed releasing action.

The serpin protein (or gene transfer vector comprising a nucleic acid encoding the serpin protein) and/or the MMP inhibitor desirably are administered in a pharmaceutical composition, which comprises a pharmaceutically acceptable carrier. Any suitable pharmaceutically acceptable carrier can be used within the context of the present invention, and such carriers are well known in the art. The choice of carrier will be determined, in part, by the particular site to which the composition is to be administered and the particular method used to administer the composition.

Suitable formulations include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the pharmaceutically acceptable carrier is a buffered saline solution. More preferably, the serpin protein, gene transfer vector, and/or MMP inhibitor for use in the present inventive methods is administered in one or more pharmaceutical compositions formulated to protect the serpin protein, gene transfer vector, or MMP inhibitor from damage prior to administration. For example, the pharmaceutical composition can be formulated to reduce loss of the gene transfer vector on devices used to prepare, store, or administer the gene transfer vector, such as glassware, syringes, or needles. The pharmaceutical composition can be formulated to decrease the light sensitivity and/or temperature sensitivity of the gene transfer vector. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition will extend the shelf life of the vector, facilitate administration, and increase the efficiency of the present inventive methods. In this regard, a pharmaceutical composition also can be formulated to enhance transduction efficiency.

In addition, one of ordinary skill in the art will appreciate that the serpin protein, gene transfer vector, and/or MMP inhibitor can be present in a composition with other therapeutic or biologically active agents. For example, therapeutic factors useful in the treatment of a particular indication can be present. For instance, if treating vision loss, hyaluronidase can be added to a composition to affect the break down of blood and blood proteins in the vitreous of the eye. Factors that control inflammation, such as ibuprofen or steroids, can be part of the composition to reduce swelling and inflammation associated with in vivo administration of the viral vector and ocular distress. Immune system suppressors can be administered in combination with the present inventive method to reduce any immune response to the vector itself or associated with an ocular disorder. Anti-angiogenic factors, such as soluble growth factor receptors, growth factor antagonists, i.e., angiotensin, and the like also can be part of the composition, as well as additional neurotrophic factors. Similarly, vitamins and minerals, anti-oxidants, and micronutrients can be co-administered. Antibiotics, i.e., microbicides and fungicides, can be present to reduce the risk of infection associated with gene transfer procedures and other disorders.

One skilled in the art will appreciate that suitable methods, i.e., invasive and noninvasive methods, of administering a gene transfer vector whereon the gene transfer vector will contact an ocular cell are available. Although more than one route can be used to administer a particular gene transfer vector, a particular route can provide a more immediate and more effective reaction than another route. Accordingly, the described routes of administration are merely exemplary and are in no way limiting.

The inventive methods are not dependent on the mode of administering the serpin protein, gene transfer vector, and/or MMP inhibitor to a mammal to achieve the desired effect. The serpin protein, gene transfer vector, and/or MMP inhibitor for use in the present inventive methods can be appropriately formulated and administered in the form of an oral tablet, injection, eye lotion, ointment, implant, and the like. The serpin protein, gene transfer vector, and MMP inhibitor can be applied, for example, orally, systemically, topically, subconjunctivally, retrobulbarly, periocularly, subretinally, suprachoroidally, intravitreally, intraocularly, intravenously, intraarterially, or intraperitoneally, or a combination thereof. In certain cases, it may be appropriate to administer multiple applications and employ multiple routes, e.g., subretinal and intravitreous, to ensure sufficient exposure of ocular cells to the gene transfer vector. Multiple applications of the gene transfer vector may also be required to achieve the desired effect.

Depending on the particular case, it may be desirable to non-invasively administer the gene transfer vector to a patient. For instance, if multiple surgeries have been performed, the patient displays low tolerance to anesthetic, or if other ocular-related disorders exist, topical administration of the gene transfer vector may be most appropriate. Topical formulations are well known to those of skill in the art. Such formulations are suitable in the context of the present invention for application to the skin. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art. The gene transfer vector can also be administered non-invasively using a needleless injection device, such as the Biojector 2000 Needle-Free Injection Management System® available from Bioject, Inc.

The gene transfer vector is preferably present in or on a device that allows controlled or sustained release of the gene transfer vector, such as an ocular sponge, meshwork, mechanical reservoir, or mechanical implant. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit, or an implant or a device comprised of a polymeric composition are particularly useful for ocular administration of the gene transfer vector. The gene transfer vector of the present inventive methods can also be administered in the form of sustained-release formulations (see, e.g., U.S. Pat. No. 5,378,475) comprising, for example, gelatin, chondroitin sulfate, a polyphosphoester, such as bis-2-hydroxyethyl-terephthalate (BHET), or a polylactic-glycolic acid.

Alternatively, the gene transfer vector can be administered using invasive procedures, such as, for instance, intravitreal injection or subretinal injection optionally preceded by a vitrectomy. Subretinal injections can be administered to different compartments of the eye, i.e., the anterior chamber. While intraocular injection is preferred, injectable compositions can also be administered intramuscularly, intravenously, and intraperitoneally. Pharmaceutically acceptable carriers for injectable compositions are well-known to those of ordinary skill in the art (see Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986)). Although less preferred, the gene transfer vector can also be administered in vivo by particle bombardment, i.e., a gene gun.

Preferably, the gene transfer vector is administered via an opthalmologic instrument for delivery to a specific region of an eye. Use of a specialized opthalmologic instrument ensures precise administration of the gene transfer vector while minimizing damage to adjacent ocular tissue. Delivery of the gene transfer vector to a specific region of the eye also limits exposure of unaffected cells, thereby reducing the risk of side effects. A preferred opthalmologic instrument is a combination of forceps and subretinal needle or sharp bent cannula.

While not particularly preferred, the gene transfer vector can be administered parenterally. Preferably, any gene transfer vector parenterally administered to a patient for the prophylactic or therapeutic treatment of an ocular-related disorder, i.e., ocular neovascularization or age-related macular degeneration, is specifically targeted to ocular cells. As discussed herein, a gene transfer vector can be modified to alter the binding specificity or recognition of a gene transfer vector for a receptor on a potential host cell. With respect to adenovirus, such manipulations can include deletion of regions of the fiber, penton, or hexon, insertions of various native or non-native ligands into portions of the coat protein, and the like. One of ordinary skill in the art will appreciate that parenteral administration can require large doses or multiple administrations to effectively deliver the gene transfer vector to the appropriate host cells.

The dose of gene transfer vector administered to an animal, particularly a human, in accordance with the present invention should be sufficient to affect the desired response in the animal over a reasonable time frame. One skilled in the art will recognize that dosage will depend upon a variety of factors, including the age, species, the pathology in question, and condition or disease state. Dosage also depends on serpin protein to be expressed, as well as the amount of tissue about to be affected or actually affected by the disease. The size of the dose also will be determined by the route, timing, and frequency of administration as well as the existence, nature, and extent of any adverse side effects that might accompany the administration of a particular gene transfer vector and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Preferably, the about 10⁶ viral particles to about 10¹² viral particles are delivered to the patient. In other words, a pharmaceutical composition can be administered that comprises a gene transfer vector concentration of from about 10⁶ particles/ml to about 10¹² particles/ml (including all integers within the range of about 10⁶ particles/ml to about 10¹² particles/ml), preferably from about 10¹⁰ particles/ml to about 10¹² particles/ml, and will typically involve the intraocular administration of from about 0.1 μl to about 100 μl of such a pharmaceutical composition per eye. When the gene transfer vector is a plasmid, preferably about 0.5 ng to about 1000 μg of DNA is administered. More preferably, about 0.1 μg to about 500 μg is administered, even more preferably about 1 μg to about 100 μg of DNA is administered. Most preferably, about 50 μg of DNA is administered per eye. Of course, other routes of administration may require smaller or larger doses to achieve a therapeutic effect. Any necessary variations in dosages and routes of administration can be determined by the ordinarily skilled artisan using routine techniques known in the art.

For protein delivery, any suitable dose of the serpin protein or therapeutic fragment or variant thereof can be administered. One of ordinary skill in the art can determine the optimum dose through means known in the art, such as through properly controlled clinical trials evaluating safety and efficacy. When the serpin protein to be administered is PEDF, a suitable dose is about 10 ng to about 100 ug depending on the indication and the site of delivery. For example, a suitable dose of the protein or therapeutic fragment or variant thereof is about 20 ng, 40 ng, 50 ng, 60 ng, 80 ng, 100 ng, 200 ng, 300 ng, 400 ng, 500 ng, 600 ng, 700 ng, 800 ng, 900 ng, 1 μg, 2 μg, 5 μg, 10 μg, 20 μg, 30 μg, 40 μg, 50 μg, 60 μg, 70 μg, 80 μg, 90 μg, or ranges thereof.

For delivery of the MMP inhibitor, any suitable dose can be administered. One of ordinary skill in the art can determine the optimum dose of the MMP inhibitor through means known in the art. The dose for the MMP inhibitor varies according to the route of delivery and the particular MMP inhibitor. For instance, an orally active form of an MMP inhibitor has a different dosing regimen than an injectable form.

In some embodiments, it is advantageous to administer two or more (i.e., multiple) doses of the serpin protein, gene transfer vector, or MMP inhibitor. The inventive methods provide for multiple applications. For example, at least two applications of a gene transfer vector can be administered to the same eye. Preferably, the multiple doses are administered while retaining gene expression above background levels. Also preferably, an ocular cell is contacted with two applications or more of the gene transfer vector within about 30 days or more. More preferably, two or more applications are administered to ocular cells of the same eye within about 90 days or more. However, three, four, five, six, or more doses can be administered in any time frame (e.g., 2, 7, 10, 14, 21, 28, 35, 42, 49, 56, 63, 70, 77, 85 or more days between doses) so long as gene expression occurs and ocular neovascularization is inhibited or ameliorated.

It also will be appreciated by one skilled in the art that a serpin protein, gene transfer vector, or MMP inhibitor can be introduced ex vivo into cells, preferably ocular cells, previously removed from a given animal, in particular a human. Such transduced autologous or homologous host cells, reintroduced into the animal or human, will express directly the serpin protein or MMP inhibitor in vivo. One ex vivo therapeutic option involves the encapsidation of infected ocular cells into a biocompatible capsule, which can be implanted in the eye or any other part of the body. One of ordinary skill in the art will understand that such cells need not be isolated from the patient, but can instead be isolated from another individual and implanted into the patient.

The gene transfer vector further can comprise one or more additional nucleic acid sequences encoding proteins other than the protein of a serpin superfamily (e.g., PEDF) or therapeutic fragment or variant thereof. For example, the gene transfer vector further can comprise one or more additional nucleic acid sequences encoding neurotrophic factors (e.g., ciliary neurotrophic factor (CNTF)), an atonal-associated peptide, an anti-angiogenic substance, another protein of the serpin superfamily, or another protease inhibitor (e.g., another MMP inhibitor).

Neurotrophic factors are thought to be responsible for the maturation of developing neurons and for maintaining adult neurons. Neurotrophic factors are divided into three subclasses: neuropoietic cytokines; neurotrophins; and the fibroblast growth factors. CNTF is exemplary of neuropoietic cytokines. CNTF promotes the survival of ciliary ganglionic neurons and supports certain neurons that are NGF-responsive. Neurotrophins include, for example, brain-derived neurotrophic factor and nerve growth factor. Other neurotrophic factors suitable for being encoded by the nucleic acid sequence of the present inventive methods include, for example, transforming growth factors, glial cell-line derived neurotrophic factor, neurotrophin 3, neurotrophin 4/5, and interleukin 1-β. Neurotrophic factors associated with angiogenesis, such as aFGF and bFGF, are less preferred. The neurotrophic factor of the present inventive method preferably is a neuronotrophic factor, e.g., a factor that enhances neuronal survival. It has been postulated that neurotrophic factors can actually reverse degradation of neurons. Such factors, conceivably, are useful in treating the degeneration of neurons associated with vision loss. Neurotrophic factors function in both paracrine and autocrine fashions, making them ideal therapeutic agents.

Atonal-associated peptides include Math1 and Hath1 and biologically active fragments of either of the foregoing. Math1 is a member of the mouse basic helix-loop-helix family of transcription factors and is homologous to the Drosophila gene atonal. Hath1 is the human counterpart of Math1. Math1 has been shown to be essential for hair development and can stimulate hair regeneration in the ear. Combining the neurotrophic properties of PEDF and the hair cell differentiation properties of an atonal-associated peptide provides a powerful tool for the treatment and research of, for example, sensory disorders. Math1 is further characterized in, for example, Bermingham et al., Science, 284, 1837-1841 (1999) and Zheng and Gao, Nature Neuroscience, 3(2), 580-586 (2000).

One or more additional nucleic acid sequences encoding therapeutic substances can encode an anti-angiogenic substance other than PEDF or a therapeutic fragment thereof. An anti-angiogenic substance is any biological factor that prevents or ameliorates neovascularization. One of ordinary skill in the art will understand that the anti-angiogenic substance can effect partial or complete prevention and amelioration of angiogenesis to achieve a therapeutic effect. An anti-angiogenic substance includes, for instance, an anti-angiogenic factor, an anti-sense molecule specific for an angiogenic factor, a ribozyme, a receptor for an angiogenic factor, and an antibody that binds a receptor for an angiogenic factor.

Anti-angiogenic factors include, for example, angiostatin, vasculostatin, endostatin, platelet factor 4, heparinase, interferons (e.g., INFα), and the like. One of ordinary skill in the art will appreciate that any anti-angiogenic factor can be modified or truncated and retain anti-angiogenic activity. As such, active fragments of anti-angiogenic factors (i.e., those fragments having biological activity sufficient to inhibit angiogenesis) are also useful for incorporation into gene transfer vector.

An anti-sense molecule specific for an angiogenic factor should generally be substantially identical to at least a portion, preferably at least about 20 continuous nucleotides, of the nucleic acid encoding the angiogenic factor to be inhibited, but need not be identical. The anti-sense nucleic acid molecule can be designed such that the inhibitory effect applies to other proteins within a family of genes exhibiting homology or substantial homology to the nucleic acid. The introduced anti-sense nucleic acid molecule also need not be full-length relative to either the primary transcription product or fully processed mRNA. Generally, higher homology can be used to compensate for the use of a shorter sequence. Furthermore, the anti-sense molecule need not have the same intron or exon pattern, and homology of non-coding segments will be equally effective. Antisense phosphorothiotac oligodeoxynucleotides (PS-ODNs) is exemplary of an anti-sense molecule specific for an angiogenic factor.

Ribozymes can be designed that specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences within anti-sense RNAs confers RNA-cleaving activity upon them, thereby increasing the activity of the constructs. The design and use of target RNA-specific ribozymes is described in Haseloff et al., Nature, 334, 585-591 (1988). Preferably, the ribozyme comprises at least about 20 continuous nucleotides complementary to the target sequence on each side of the active site of the ribozyme.

Receptors specific for angiogenic factors inhibit neovascularization by sequestering growth factors away from functional receptors capable of promoting a cellular response. For example, soluble VEGF-R1 (flt-1), VEGF-R2 (flk/kdr), and VEGF-R3 (flt-4) receptors, as well as VEGF-receptor chimeric proteins, compete with VEGF receptors on vascular endothelial cells to inhibit endothelial cell growth (Aiello, PNAS, 92, 10457 (1995)). Preferably, the viral vector of the present invention comprises at least one nucleic acid sequence encoding soluble flt receptor in addition to the nucleic acid sequence encoding PEDF or a therapeutic fragment thereof. Receptors specific for angiogenic factors, in particular the soluble flt receptor, and use thereof to inhibit angiogenesis is further described in Kendall et al., PNAS, 90(22), 10705-10709 (1993), Kong et al., Human Gene Therapy, 9, 823-833 (1988), and International Patent Application WO 94/21679. Also contemplated are growth factor-specific antibodies and fragments thereof (e.g., Fab, F(ab′)2, and Fv) that neutralize angiogenic factors (e.g., VEGF) or bind receptors for angiogenic factors.

One of ordinary skill in the art will appreciate that any of a number of gene transfer vectors known in the art are suitable for use in the present inventive methods. Examples of suitable gene transfer vectors include, for instance, naked DNA, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, AAV-adenoviral chimeric vectors, HIV virus-based vectors, and adenovirus-based vectors. Any of these gene transfer vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Specifically, vectors, such as those described in Semkova et al., PNAS, 99(2), 13090-13095 (2002); Miyazaki et al., Gene Ther., 10(17), 1503-11 (2003). Auricchio et al., Mol. Ther., 6(4), 490-494 (2002); Mori et al., Inves. Opthalmol. Vis. Sci., 43(6), 1994-2000 (2002); and Nomura et al., Dev. Neurosci., 23(2), 145-52 (2001), are envisioned for use with the invention.

Plasmids, genetically engineered circular double-stranded DNA molecules, can be designed to contain an expression cassette for delivery of the nucleic acid sequence. Although plasmids were the first vector described for administration of therapeutic nucleic acids, the level of transfection efficiency is poor compared with other techniques. By complexing the plasmid with liposomes, the efficiency of gene transfer in general is improved. While the liposomes used for plasmid-mediated gene transfer strategies have various compositions, they are typically synthetic cationic lipids. Advantages of plasmid-liposome complexes include their ability to transfer large pieces of DNA encoding a therapeutic nucleic acid and their relatively low immunogenicity.

Plasmids are often used for short-term expression. However, a plasmid construct can be modified to obtain prolonged expression. The inverted terminal repeats (ITR) of parvovirus, in particular adeno-associated virus (AAV), are responsible for the high-level persistent nucleic acid expression often associated with AAV (see, for example, U.S. Pat. No. 6,165,754). Accordingly, the gene transfer vector can be a plasmid comprising native parvovirus ITRs to obtain prolonged and substantial expression of the serpin protein. While plasmids are suitable for use in the present inventive methods, preferably the gene transfer vector is a viral vector.

AAV vectors are viral vectors of particular interest for use in gene therapy protocols. AAV is a DNA virus, which is not known to cause human disease. AAV requires co-infection with a helper virus (i.e., an adenovirus or a herpes virus), or expression of helper genes, for efficient replication. AAV vectors used for administration of a therapeutic nucleic acid have approximately 96% of the parental genome deleted, such that only the terminal repeats (ITRs), which contain recognition signals for DNA replication and packaging, remain. This eliminates immunologic or toxic side effects due to expression of viral genes. In addition, delivering the AAV rep protein enables integration of the AAV vector comprising AAV ITRs into a specific region of genome, if desired. Host cells comprising an integrated AAV genome show no change in cell growth or morphology (see, for example, U.S. Pat. No. 4,797,368). Although efficient, the need for helper virus or helper genes can be an obstacle for widespread use of this vector.

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. When employing pathogenic retroviruses, e.g., human immunodeficiency virus (HIV) or human T-cell lymphotrophic viruses (HTLV), care must be taken in altering the viral genomic to eliminate toxicity. A retroviral vector can additionally be manipulated to render the virus replication-incompetent. As such, retroviral vectors are thought to be particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating atrophic forms of ocular-related disease.

HSV-based viral vectors are suitable for use as a gene transfer vector to introduce nucleic acids into ocular cells. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. Of course, this ability is also a disadvantage in terms of short-term treatment regimens. For a description of HSV-based vectors appropriate for use in the present inventive methods, see, for example, U.S. Pat. Nos. 5,837,532; 5,846,782; 5,849,572; and 5,804,413 and International Patent Applications WO 91/02788, WO 96/04394, WO 98/15637, and WO 99/06583.

Adenovirus (Ad) is a 36 kb double-stranded DNA virus that efficiently transfers DNA in vivo to a variety of different target cell types. For use in the present inventive methods, the virus is preferably made replication deficient by deleting select genes required for viral replication. The expendable E3 region is also frequently deleted to allow additional room for a larger DNA insert. The vector can be produced in high titers and can efficiently transfer DNA to replicating and non-replicating cells. The newly transferred genetic information remains epi-chromosomal, thus eliminating the risks of random insertional mutagenesis and permanent alteration of the genotype of the target cell. However, if desired, the integrative properties of AAV can be conferred to adenovirus by constructing an AAV-Ad chimeric vector. For example, the AAV ITRs and nucleic acid encoding the Rep protein incorporated into an adenoviral vector enables the adenoviral vector to integrate into a mammalian cell genome. Therefore, AAV-Ad chimeric vectors are an interesting option for use in the present invention.

Preferably, the gene transfer vector of the present inventive methods is a viral vector; more preferably, the gene transfer vector is an adenoviral vector. In the context of the present invention, the adenoviral vector can be derived from any serotype of adenovirus. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotype 2, 5 or 9. However, non-group C adenoviruses can be used to prepare replication-deficient adenoviral gene transfer vectors for delivery of the serpin protein. Preferred adenoviruses used in the construction of non-group C adenoviral gene transfer vectors include Ad12 (group A), Ad7 (group B), Ad30 and Ad36 (group D), Ad4 (group E), and Ad41 (group F). Non-group C adenoviral vectors, methods of producing non-group C adenoviral vectors, and methods of using non-group C adenoviral vectors are disclosed in, for example, U.S. Pat. Nos. 5,801,030; 5,837,511; and 5,849,561 and International Patent Applications WO 97/12986 and WO 98/53087.

The adenoviral vector is preferably deficient in at least one gene function required for viral replication, thereby resulting in a “replication-deficient” adenoviral vector. Preferably, the adenoviral vector is deficient in at least one essential gene function of the E1 region of the adenoviral genome required for viral replication. In addition to a deficiency in the E1 region, the recombinant adenovirus can also have a mutation in the major late promoter (MLP). The mutation in the MLP can be in any of the MLP control elements such that it alters the responsiveness of the promoter, as discussed in International Patent Application WO 00/00628. More preferably, the vector is deficient in at least one essential gene function of the E1 region and at least part of the E3 region (e.g., an Xba I deletion of the E3 region). With respect to the E1 region, the adenoviral vector can be deficient in at least part of the E1a region and at least part of the E1b region. Preferably, the adenoviral vector is “multiply deficient,” meaning that the adenoviral vector is deficient in one or more essential gene functions required for viral replication in each of two or more regions. For example, the aforementioned E1-deficient or E1-, E3-deficient adenoviral vectors can be further deficient in at least one essential gene of the E4 region. Adenoviral vectors deleted of the entire E4 region can elicit lower host immune responses.

Alternatively, the adenoviral vector lacks all or part of the E1 region and all or part of the E2 region. However, adenoviral vectors lacking all or part of the E1 region, all or part of the E2 region, and all or part of the E3 region also are contemplated herein. In one embodiment, the adenoviral vector lacks all or part of the E1 region, all or part of the E2 region, all or part of the E3 region, and all or part of the E4 region. Suitable replication-deficient adenoviral vectors are disclosed in U.S. Pat. Nos. 5,851,806 and 5,994,106 and International Patent Applications WO 95/34671 and WO 97/21826. For example, suitable replication-deficient adenoviral vectors include those with at least a partial deletion of the E1a region, at least a partial deletion of the E1b region, at least a partial deletion of the E2a region, and at least a partial deletion of the E3 region. Alternatively, the replication-deficient adenoviral vector can have at least a partial deletion of the E1 region, at least a partial deletion of the E3 region, and at least a partial deletion of the E4 region. Such multiply-deficient viral vectors are particularly useful in that such vectors can accept large inserts of exogenous DNA. Indeed, adenoviral amplicons, an example of a multiply-deficient adenoviral vector which comprises only those genomic sequences required for packaging and replication of the viral genome, can accept inserts of approximately 36 kb.

It should be appreciated that the deletion of different regions of the adenoviral vector can alter the immune response of the mammal. In particular, deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509. Such modifications are useful for long-term treatment of persistent ocular disorders.

Similarly, the coat protein of a viral vector, preferably an adenoviral vector, can be manipulated to alter the binding specificity or recognition of a virus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by a viral vector or enable targeting of a viral vector to a specific cell type. For example, in one embodiment, the gene transfer vector is a viral vector comprising a chimeric coat protein (e.g., a fiber, hexon pIX, pIIIa, or penton protein), which differs from the wild-type (i.e., native) coat protein by the introduction of a normative amino acid sequence, preferably at or near the carboxyl terminus. Preferably, the normative amino acid sequence is inserted into or in place of an internal coat protein sequence. One of ordinary skill in the art will understand that the normative amino acid sequence can be inserted within the internal coat protein sequence or at the end of the internal coat protein sequence. The resultant chimeric viral coat protein is able to direct entry into cells of the viral, i.e., adenoviral, vector comprising the coat protein that is more efficient than entry into cells of a vector that is identical except for comprising a wild-type viral coat protein rather than the chimeric viral coat protein. Preferably, the chimeric virus coat protein binds a novel endogenous binding site present on the cell surface that is not recognized, or is poorly recognized by a vector comprising a wild-type coat protein. One direct result of this increased efficiency of entry is that the virus, preferably, the adenovirus, can bind to and enter numerous cell types which a virus comprising wild-type coat protein typically cannot enter or can enter with only a low efficiency.

In another embodiment of the present invention, the gene transfer vector is a viral vector comprising a chimeric virus coat protein not selective for a specific type of eukaryotic cell. The chimeric coat protein differs from the wild-type coat protein by an insertion of a normative amino acid sequence into or in place of an internal coat protein sequence. In this embodiment, the chimeric virus coat protein efficiently binds to a broader range of eukaryotic cells than a wild-type virus coat, such as described in International Patent Application WO 97/20051.

Specificity of binding of an adenovirus to a given cell can also be adjusted by use of an adenovirus comprising a short-shafted adenoviral fiber gene, as discussed in U.S. Pat. No. 5,962,311. Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of an adenovirus comprising a short-shafted fiber enables targeting of the adenovirus to a desired cell-surface receptor by the introduction of a normative amino acid sequence either into the penton base or the fiber knob.

Of course, the ability of a viral vector to recognize a potential host cell can be modulated without genetic manipulation of the coat protein. For instance, complexing an adenovirus with a bispecific molecule comprising a penton base-binding domain and a domain that selectively binds a particular cell surface binding site enables one of ordinary skill in the art to target the vector to a particular cell type.

Suitable modifications to a viral vector, specifically an adenoviral vector, are described in U.S. Pat. Nos. 5,559,099; 5,731,190; 5,712,136; 5,770,442; 5,846,782; 5,926,311; 5,965,541; 6,057,155; 6,127,525; and 6,153,435 and International Patent Applications WO 96/07734, WO 96/26281, WO 97/20051, WO 98/07865, WO 98/07877, WO 98/54346, and WO 00/15823. Similarly, it will be appreciated that numerous gene transfer vectors are available commercially. Construction of gene transfer vectors is well understood in the art. Adenoviral vectors can be constructed and/or purified using the methods set forth, for example, in U.S. Pat. No. 5,965,358 and International Patent Applications WO 98/56937, WO 99/15686, and WO 99/54441. Adeno-associated viral vectors can be constructed and/or purified using the methods set forth, for example, in U.S. Pat. No. 4,797,368 and Laughlin et al., Gene, 23, 65-73 (1983).

The selection of gene transfer vectors for use for use in the present inventive method will depend on a variety of factors such as, for example, the host, immunogenicity of the vector, the desired duration of protein production, and the like. As each type of gene transfer vector has distinct properties, a researcher has the freedom to tailor the present inventive method to any particular situation. Moreover, more than one type of gene transfer vector can be used to deliver the nucleic acid sequence to the desired tissue.

Preferably, the nucleic acid sequence is operably linked to regulatory sequences necessary for expression, i.e., a promoter. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the present invention to provide for transcription of the nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

A comparison of promoter sequences that function in eukaryotes has revealed conserved sequence elements. Generally, eukaryotic promoters transcribed by RNA polymerase II are typified by a “TATA box” centered at approximately position −25, which appears to be essential for accurately positioning the start of transcription. The TATA box directs RNA polymerase to begin transcribing approximately 30 base pairs (bp) downstream in mammalian systems. The TATA box functions in conjunction with at least two other upstream sequences located about 40 bp and 110 bp upstream of the start of transcription. Typically, a so-called “CCAAT box” serves as one of the two upstream sequences, and the other often is a GC-rich segment. The CCAAT homology can reside on different strands of the DNA. The upstream promoter element also can be a specialized signal such as one of those which have been described in the art and which appear to characterize a certain subset of genes.

To initiate transcription, the TATA box and the upstream sequences are each recognized by regulatory proteins that bind to these sites, and activate transcription by enabling RNA polymerase II to bind the DNA segment and properly initiate transcription. Whereas base changes outside the TATA box and the upstream sequences have little effect on levels of transcription, base changes in either of these elements substantially lower transcription rates (see, e.g., Myers et al., Science, 229, 242-247 (1985); McKnight et al., Science, 217, 316-324 (1982)). The position and orientation of these elements relative to one another, and to the start site, are important for the efficient transcription of some, but not all, coding sequences. For instance, some promoters function well in the absence of any TATA box. Similarly, the necessity of these and other sequences for promoters recognized by RNA polymerase I or III, or other RNA polymerases, can differ.

Accordingly, promoter regions can vary in length and sequence and can further encompass one or more DNA binding sites for sequence-specific DNA binding proteins and/or an enhancer or silencer. Enhancers and/or silencers can similarly be present on a nucleic acid sequence outside of the promoter per se.

The present invention preferentially employs a viral promoter. Suitable viral promoters are known in the art and include, for instance, cytomegalovirus (CMV) promoters, such as the CMV immediate-early promoter, promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like. Preferably, the viral promoter is an adenoviral promoter, such as the Ad2 or Ad5 major late promoter and tripartite leader, a CMV promoter, or an RSV promoter.

Many of the above-described promoters are constitutive promoters. Instead of being a constitutive promoter, the promoter can be an inducible promoter, i.e., a promoter that is up- and/or down-regulated in response to appropriate signals. For instance, the regulatory sequences can comprise a hypoxia driven promoter, which is active when the ocular neovascularization or age-related macular degeneration is associated with hypoxia. Other examples of suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. The promoter sequence that regulates expression of the nucleic acid sequence can contain at least one heterologous regulatory sequence responsive to regulation by an exogenous agent. The regulatory sequences are preferably responsive to exogenous agents such as, but not limited to, drugs, hormones, or other gene products. For example, the regulatory sequences, e.g., promoter, preferably are responsive to glucocorticoid receptor-hormone complexes, which, in turn, enhance the level of transcription of the serpin protein or a therapeutic fragment or variant thereof.

Preferably, the regulatory sequences comprise a tissue-specific promoter, i.e., a promoter that is preferentially activated in a given tissue and results in expression of a gene product in the tissue where activated. A typically used tissue-specific promoter is a myocyte-specific promoter. A promoter exemplary of a myocyte-specific promoter is the myosin light-chain 1A promoter. A tissue specific promoter for use in the vector can be chosen by the ordinarily skilled artisan based upon the target tissue or cell-type. Preferred tissue-specific promoters for use in the present inventive methods are specific to ocular tissue, such as a rhodopsin promoter. Examples of rhodopsin promoters include, but are not limited to, a GNAT cone-transducing alpha-subunit gene promoter or an interphotoreceptor retinoid binding protein promoter.

One of ordinary skill in the art will appreciate that each promoter drives transcription, and, therefore, protein expression, differently with respect to time and amount of protein produced. For example, the CMV promoter is characterized as having peak activity shortly after transduction, i.e., about 24 hours after transduction, then quickly tapering off. On the other hand, the RSV promoter's activity increases gradually, reaching peak activity several days after transduction, and maintains a high level of activity for several weeks. Indeed, sustained expression driven by an RSV promoter has been observed in all cell types studied, including, for instance, liver cells, lung cells, spleen cells, diaphragm cells, skeletal muscle cells, and cardiac muscle cells. Thus, a promoter can be selected for use in the methods of the present invention by matching its particular pattern of activity with the desired pattern and level of expression of the serpin protein. Alternatively, a hybrid promoter can be constructed which combines the desirable aspects of multiple promoters. For example, a CMV-RSV hybrid promoter combining the CMV promoter's initial rush of activity with the RSV promoter's high maintenance level of activity would be especially preferred for use in many embodiments of the present inventive method. It is also possible to select a promoter with an expression profile that can be manipulated by an investigator.

Also preferably, the gene transfer vector comprises a nucleic acid encoding a cis-acting factor, wherein the cis-acting factor modulates the expression of the nucleic acid sequence. Preferably, the cis-acting factor comprises matrix attachment region (MAR) sequences (e.g., immunoglobulin heavy chain (Jenunwin et al., Nature, 385(16), 269 (1997)), apolipoprotein B, or locus control region (LCR) sequences, among others. MAR sequences have been characterized as DNA sequences that associate with the nuclear matrix after a combination of nuclease digestion and extraction (Bode et al., Science, 255(5041), 195-197 (1992)). MAR sequences are often associated with enhancer-type regulatory regions and, when integrated into genomic DNA, MAR sequences augment transcriptional activity of adjacent nucleotide sequences. It has been postulated that MAR sequences play a role in controlling the topological state of chromatin structures, thereby facilitating the formation of transcriptionally-active complexes. Similarly, it is believed LCR sequences function to establish and/or maintain domains permissive for transcription. Many LCR sequences give tissue specific expression of associated nucleic acid sequences. Addition of MAR or LCR sequences to the gene transfer vector can further enhance expression the serpin protein.

With respect to promoters, nucleic acid sequences, selectable markers, and the like, located on an gene transfer vector, such elements can be present as part of a cassette, either independently or coupled. In the context of the present invention, a “cassette” is a particular base sequence that possesses functions which facilitate subcloning and recovery of nucleic acid sequences (e.g., one or more restriction sites) or expression (e.g., polyadenylation or splice sites) of particular nucleic acid sequences.

Construction of an exogenous nucleic acid operably linked to regulatory sequences necessary for expression is well within the skill of the art (see, for example, Sambrook et al., Molecular Cloning: A Laboratory Manual, 2d ed. (1989)). With respect to the expression of nucleic acid sequences according to the present invention, the ordinary skilled artisan is aware that different genetic signals and processing events control levels of nucleic acids and proteins/peptides in a cell, such as, for instance, transcription, mRNA translation, and post-transcriptional processing. Transcription of DNA into RNA requires a functional promoter, as described herein.

Protein expression is dependent on the level of RNA transcription that is regulated by DNA signals, and the levels of DNA template. Similarly, translation of mRNA requires, at the very least, an AUG initiation codon, which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J. Molec. Biol., 196, 947-950 (1987)). Also, successful expression of an exogenous nucleic acid in a cell can require post-translational modification of a resultant protein. Thus, production of a protein can be affected by the efficiency with which DNA (or RNA) is transcribed into mRNA, the efficiency with which mRNA is translated into protein, and the ability of the cell to carry out post-translational modification. These are all factors of which the ordinary skilled artisan is aware and is capable of manipulating using standard means to achieve the desired end result.

Along these lines, to optimize protein production, preferably the nucleic acid sequence further comprises a polyadenylation site following the coding region of the nucleic acid sequence. Also, preferably all the proper transcription signals (and translation signals, where appropriate) will be correctly arranged such that the nucleic acid sequence will be properly expressed in the cells into which it is introduced. If desired, the nucleic acid sequence also can incorporate splice sites (i.e., splice acceptor and splice donor sites) to facilitate mRNA production. Moreover, if the nucleic acid sequence encodes a protein or peptide, which is a processed or secreted protein or acts intracellularly, preferably the nucleic acid sequence further comprises the appropriate sequences for processing, secretion, intracellular localization, and the like.

The invention also provides a method of treating a disease in a mammal, which method comprises administering to the mammal (a) a gene transfer vector comprising a nucleic acid sequence encoding protein of a serpin superfamily or variant thereof and (b) an inhibitor of a matrix metalloprotease (MMP), wherein the nucleic acid sequence encoding the protein of a serpin superfamily or variant thereof is expressed, thereby treating the disease in the mammal.

Similarly, the invention encompasses a method of treating a disease in a mammal, which method comprises administering to the mammal (a) a protein of a serpin superfamily or variant thereof and (b) an inhibitor of a matrix metalloprotease (MMP), thereby treating the disease in the mammal.

The mammal to be treated can be any suitable mammal, including, but not limited to, mice, rats, cats, dogs, guinea pigs, hamsters, rabbits, cats, dogs, pigs, cows, horses, primates, and humans. Preferably, the mammal is a human.

The disease to be treated can be any suitable disease. Suitable diseases include, but are not limited to, ocular-related diseases, ear-related diseases, joint diseases, arthritis, atherosclerosis, wound healing, diabetic neuropathy, geographic atrophy, glaucoma, Leber's congenital amaurosis, surgical procedures, cancer, diseases of the nervous system, and spinal injury/trauma. Preferably, the disease is an ocular-related disease, ear-related disease, joint disease, cancer, or a disease of the nervous system. Ocular-related diseases include neovascularization of the choroid, neovascularization of the retina, neovascularization of the cornea, central and/or branched retinal vein occlusion, diabetic retinopathy, diabetic macular edema, and retinal degenerative diseases (e.g., retinitis pigmentosa, Usher syndrome, and age-related macular degeneration (e.g., wet or dry)). Diseases of the nervous system for use in the inventive method include sleep disorders, amyotrophic lateral sclerosis (Lou Gehrig's Disease), Alzheimer's Disease, epilepsy, multiple sclerosis, Parkinson's Disease, peripheral neuropathies, schizophrenia, depression, anxiety, spinal cord injury, traumatic brain injury, stroke, and inflammatory pain. One of ordinary skill in the art will appreciate that the progression of many cancers depends upon the induction of neovascularization to support tumors and metastases. Tumors often express high levels of proteases that decrease serpin protein (e.g., PEDF) activity. As members of the serpin superfamily, such as PEDF, have been shown to have anti-tumor activity, the use of stabilized serpin protein (e.g., PEDF) using sequence alterations or inhibitors of proteases improves the anti-tumor activity of serpin protein (e.g., PEDF) delivery. Thus, the inventive method can be used to treat any suitable cancer, such as, for example, retinoblastoma.

The finding that proteins of the serpin superfamily (e.g., PEDF) are sensitive to cleavage by certain proteases (e.g., trypsin, chymotrypsin, and MMPs, such as MMP-2 and MMP-9) allows the identification of sites within the sequence of the protein of the serpin superfamily (e.g., PEDF) that, if changed, will lead to a more stable and active protein. Such sites will be present in the region analogous to the protease sensitive loop of other proteins of the serpin superfamily, as well. Alteration of such protease sensitive sites will reduce the breakdown of the serpin protein, stabilize the protein in vivo, and provide for higher concentrations of the serpin proteins, and therefore, more activity for anti-angiogenic or neuroprotective applications. For example, when the altered, stabilized serpin proteins are encoded by a gene transfer vector, the gene transfer vector delivers the altered serpin protein, which has improved activity due to the increased stability of the serpin protein resulting in higher anti-anti-angiogenic and/or neuroprotective abilities. Similarly, altered and stabilized serpin proteins derived from recombinant vectors are useful for protein therapy applications. Certain serpin proteins, such as PEDF, have a relatively short half-life (e.g., following administration to the eye). Stabilized serpin proteins (e.g., PEDF) have longer persistence (e.g., following injection into the eye) and, therefore, more activity and efficacy.

Accordingly, the invention encompasses a method of inhibiting angiogenesis within a tissue, which method comprises contacting the tissue with (a) a protein of a serpin superfamily or therapeutic fragment or variant thereof or (b) a gene transfer vector comprising a nucleic acid sequence encoding a protein of a serpin superfamily or therapeutic fragment or variant thereof, wherein the nucleic acid sequence encoding the protein of the serpin superfamily or therapeutic fragment or variant thereof is expressed, wherein the protein or nucleic acid encoding the protein comprises at least one mutation which renders the protein of the serpin superfamily or therapeutic fragment or variant thereof resistant to cleavage by a matrix metalloprotease (MMP), and angiogenesis within the tissue is inhibited.

The invention also encompasses a method of promoting neuron protection within a tissue, which method comprises contacting the tissue with (a) a protein of a serpin superfamily or therapeutic fragment or variant thereof or (b) a gene transfer vector comprising a nucleic acid sequence encoding a protein of a serpin superfamily or therapeutic fragment or variant thereof, wherein the nucleic acid sequence encoding the protein of the serpin superfamily or therapeutic fragment or variant thereof is expressed, wherein the protein or nucleic acid encoding the protein comprises at least one mutation which renders the protein of the serpin superfamily or therapeutic fragment or variant thereof resistant to cleavage by a matrix metalloprotease (MMP), whereby neuron protection within is promoted.

Similarly, the invention is directed to a method of treating a disease in a mammal, which method comprises administering to the mammal (a) a protein of a serpin superfamily or therapeutic fragment or variant thereof or (b) a gene transfer vector comprising a nucleic acid sequence encoding a protein of a serpin superfamily or therapeutic fragment or variant thereof, wherein the nucleic acid sequence encoding the protein of the serpin superfamily or therapeutic fragment or variant thereof is expressed, wherein the protein or nucleic acid encoding the protein comprises at least one mutation which renders the protein of the serpin superfamily or therapeutic fragment or variant thereof resistant to cleavage by a matrix metalloprotease (MMP), whereby the disease is treated in the mammal.

The mutation in the protein or nucleic acid encoding the protein maintains the desired serpin protein activity (anti-angiogenesis, neuron protection, disease treatment), but prevents MMP-mediated degradation of the serpin protein. By preventing MMP-mediated degradation, the duration of the serpin protein activity can be extended within the tissue to be treated.

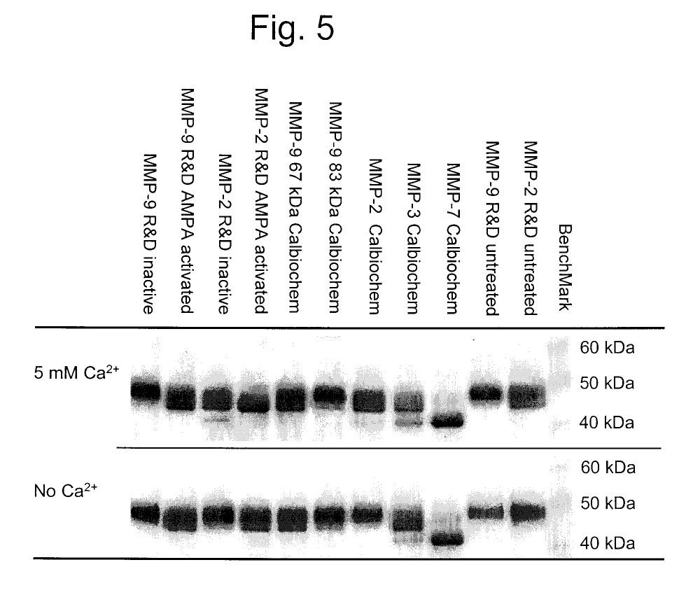

Preferably, the region to be mutated encodes (wherein the sequence to be mutated is a nucleic acid sequence) or comprises (wherein the sequence to be mutated is an amino acid sequence) the amino acid sequence of L A A A * V S N F (SEQ ID NO: 1), wherein the MMP would typically cleave between the A and V as indicated by the asterisk; and/or Q P A H * L T F P (SEQ ID NO: 2), wherein the MMP would typically cleave between the H and L as indicated by the asterisk. The N terminal domain of PEDF contains L A A A V S N F (SEQ ID NO: 1) and the Reactive Center Loop of PEDF contains Q P A H L T F P (SEQ ID NO: 2) (see FIG. 5). Other serpin family members contain analogous sequences that can be mutated to prevent cleavage by MMP inhibitors, such as MMP-9. Standard recombinant DNA techniques for creating mutations are described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

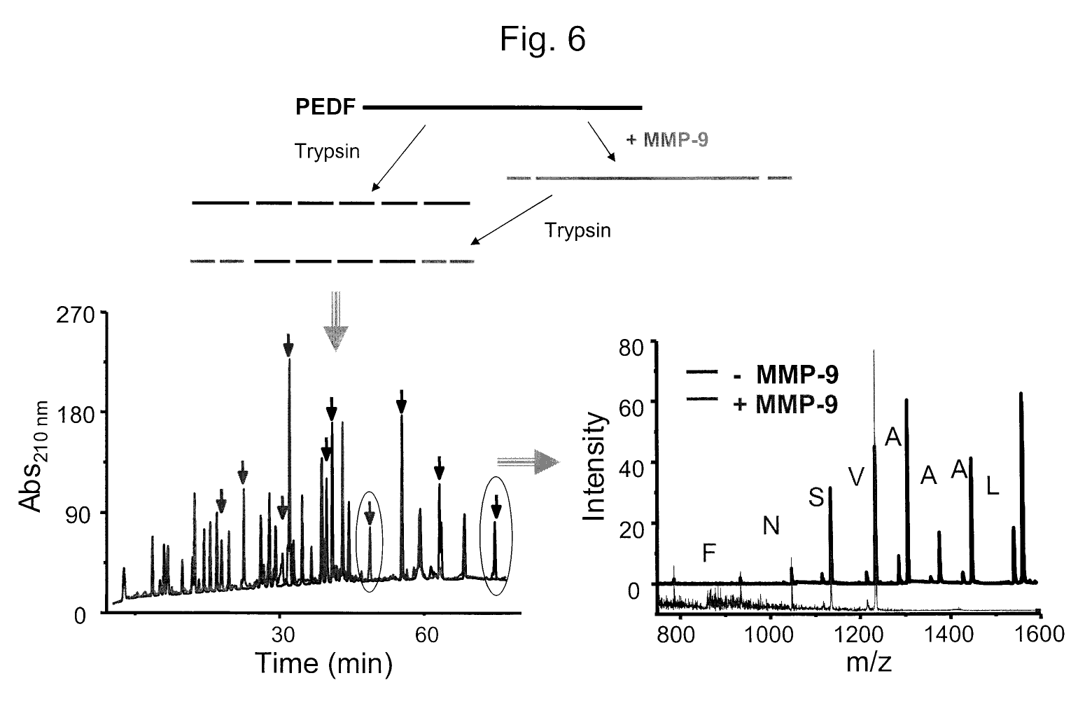

Additional preferred sequences to be mutated encode or comprise X P R S/T * Hy S/T X X (SEQ ID NO: 3), which is the consensus sequence of the cleavage site of MMP-9, wherein X denotes any amino acid, Hy denotes a hydrophobic amino acid (e.g., A, C, I, L, M, F, P, W, Y, or V), and cleavage occurs between S/T and Hy as indicated by the asterisk (see FIGS. 5 and 6). The consensus sequence for MMP-9 is not exact, so an additional “consensus sequence” is X Xₐ X_{b} X_{c} * Hy X_{d} Xₑ X (SEQ ID NO: 4), wherein X denotes any amino acid; Xₐ can be A, G, or L; X_{b} can be R, L, or A; X_{c} can be E, K, H, A, or R, X_{d} can be L, K, G, or A, Xₑ can be G, D, A, or N; and Hy denotes a hydrophobic amino acid as described above (see FIG. 5). Even this additional “consensus sequence” does not encompass all of the potential cleavage sites described in FIG. 6. Those of ordinary skill in the art would recognize how to identify MMP cleavage sites using techniques known in the art and those described in Example 2.

The following examples further illustrate the invention but, of course, should not be construed as in any way limiting its scope.

### EXAMPLE 1

This example demonstrates the effect of an MMP on the activity of a serpin protein.

A mouse aortic ring assay (MARA) was prepared essentially as described in Masson et al., Biol. Proced. Online 4(1), 24-31 (2002). Briefly, aortas from C57BL/6 mice were harvested and cleaned, and the aortas were cut into rings of about 0.5 to 0.8 mm. The rings were placed on BD Matrigel™ Matrix, which is a solubulized basement membrane preparation extracted from EHS mouse sarcoma, a tumor rich in ECM proteins. Media above the rings contained 25 ng/mL of FGF and 2.5 ng/mL of VEGF. The experimental wells contained 0 (growth factor (GF) control), 0.1 nm, 1 nM, 10 nM, 100 nM, or 1000 nM of human recombinant PEDF (hrPEDF). As an additional control, the experimental preparation was repeated without the addition of PEDF, FGF, or VEGF.

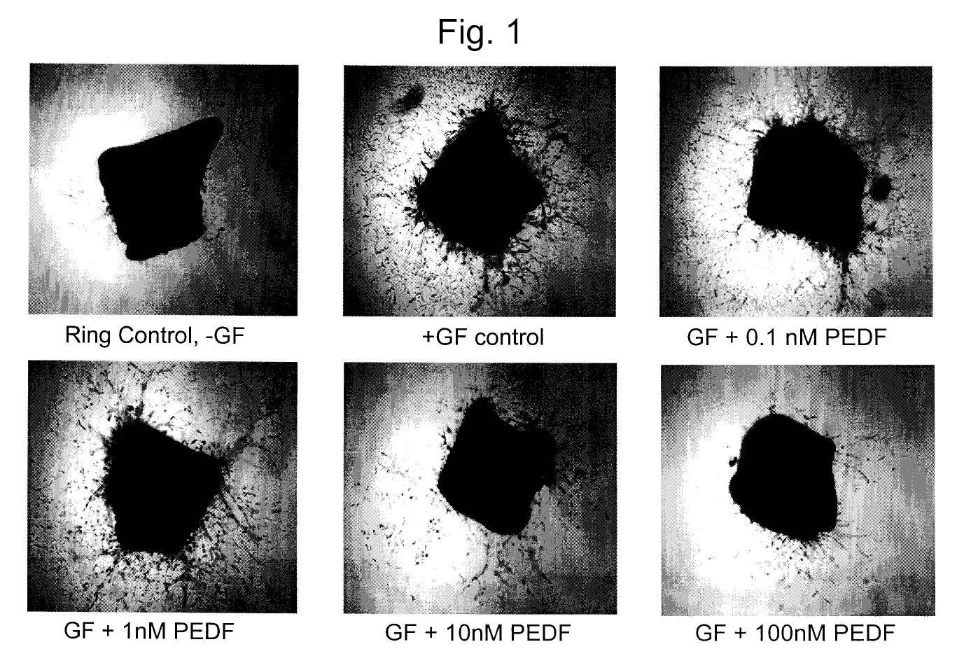

FIG. 1 illustrates the results approximately 5 days after of the beginning of the mARA (on Day 5). Without the growth factors (−GF), no endothelial cell growth was observed. With only the growth factors (+GF; GF control) and no PEDF, copious cell growth was observed. With the addition of the growth factors and PEDF, the cell growth observed in the GF control was increasingly reduced corresponding to increasing PEDF concentrations.

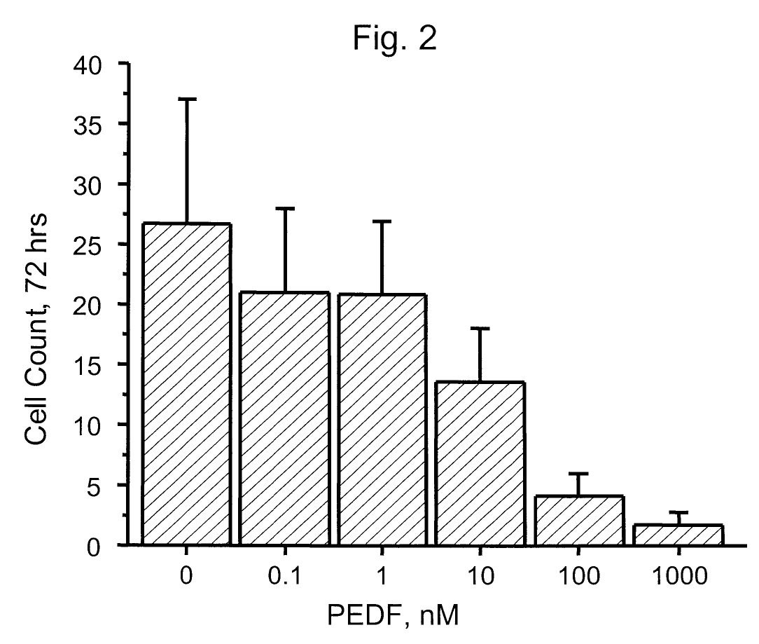

At Day 3, the number of cells could be easily counted. FIG. 2 illustrates the endothelial cell count at 72 hours after addition of 0, 0.1, 1, 10, 100, and 1000 nM PEDF. As was observed in FIG. 1, increasing amounts of PEDF resulted in lower cell counts.

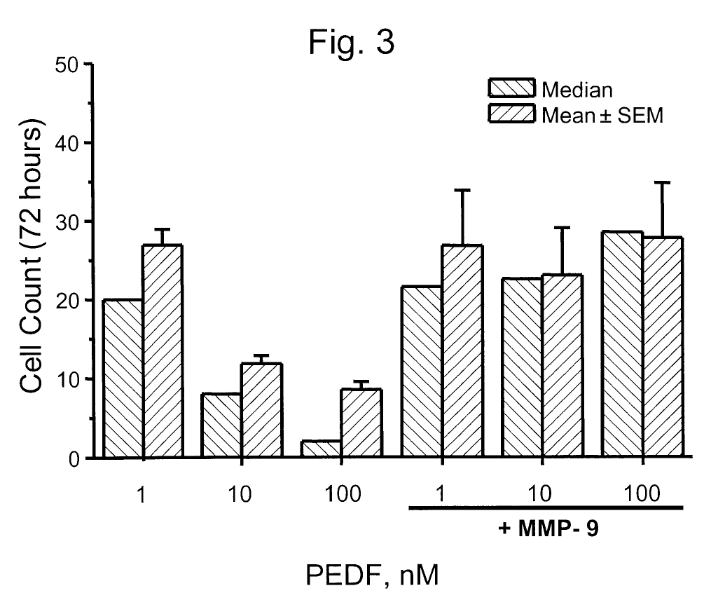

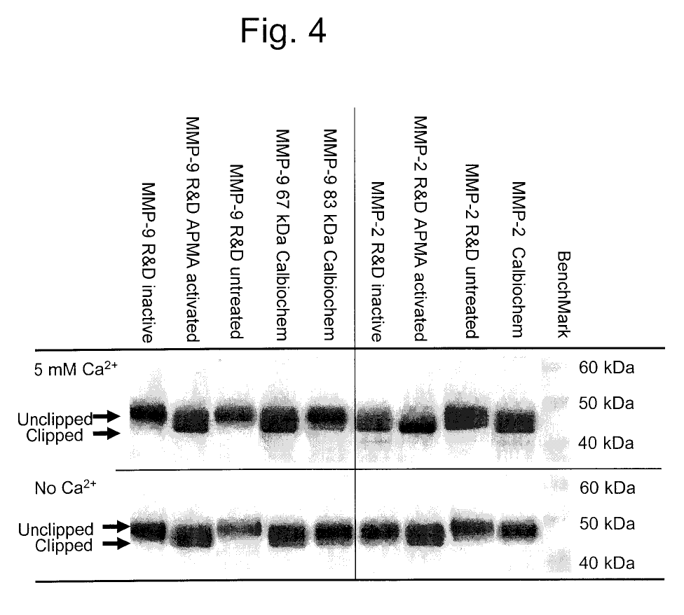

The mARA was repeated as described above with the addition of hrPEDF that was pre-digested human recombinant MMP-9 (hrMMP-9). A diluted concentration of MMP-9 (0.01 μg of MMP-9 per μg of PEDF) was added to a vial containing PEDF and incubated overnight at 28° C. A PEDF control vial was processed in the same manner, but without the addition of MMP-9. As illustrated in FIG. 3, the addition of MMP-9-treated PEDF resulted in a significant increase in the number of endothelial cells for PEDF concentrations of 10 and 100 nM when compared to the untreated controls at the same concentrations. For example, with the addition of 10 nM PEDF, the median cell count was about 8 and the mean (±standard of the mean (SEM)) cell count was about 12. In contrast, with the addition of 10 nM MMP-9-treated PEDF to the experimental wells, the median and mean (±SEM) cell count were approximately 22 and 23, respectively.

An even greater difference was observed with the addition of 100 nM PEDF, wherein the median cell count was about 2 and the mean (±SEM) cell count was about 8 for the untreated controls. With the addition of 100 nM PEDF pre-digested with MMP-9 to the experimental wells, the median cell count was about 29 and the mean (±SEM) was about 28. Thus, pre-digestion of PEDF with MMP-9 significantly increased the number of cells observed in the mARA when compared to untreated PEDF controls.

This example demonstrates that digestion of a serpin protein, such as PEDF, with an MMP, such as MMP-9, results in a decrease of the anti-angiogenic activity of the serpin protein.

### EXAMPLE 2

This example demonstrates the cleavage of a serpin protein by an MMP.

5 μg purified PEDF was incubated with MMP-2, MMP-3, MMP-7, and MMP-9 for 2 hours at 37° C. in TNTZ buffer with and without 5 mM CaCl₂. MMP-2 and MMP-9 were obtained from two different manufacturers (i.e., Calbiochem, San Diego, Calif. and R&D Systems, Minneapolis, Minn.).

Treatment of 5 μg of purified hrPEDF with 0.5 μg activated MMP-9, activated MMP-2, and MMP-7 for two hours at 37° C. resulted in the generation of multiple PEDF fragments that were observed by SDS-PAGE. These fragments had estimated relative molecular weights of 44 and 42 KDa (see FIGS. 4 and 5).

To determine the cleavage sites of MMP-9 on PEDF, 5 μg of hrPEDF was treated with (a) 0.25 μg trypsin for 16 hours at 37° C. or (b) 0.5 μg MMP-9 for 2 hours at 37° C. followed by 0.5 μg trypsin for 16 hours at 37° C. The resulting peptides were separated by reversed-phase high-pressure liquid chromatography (RP-HPLC) on a Vydac C4 column using a 0.1% trifluoroacetic acid/acetonitrile buffer system and a linear elution gradient. Differences in fragment elution were compared for both samples (see FIG. 6). Fragments present in trypsin-treated hrPEDF, but not trypsin/MMP-9-treated hrPEDF contained MMP-9 cleavage sites. Sequencing of these fragments allowed the identification of the specific sequence of the MMP-9 cleavage sites (see FIG. 6).

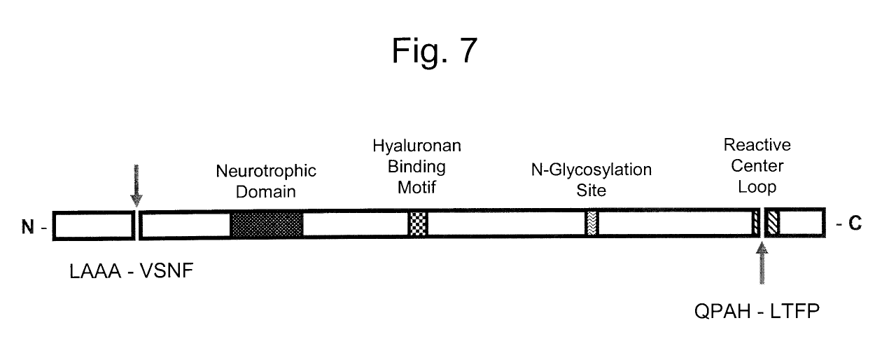

At least two MMP-9 cleavage sites on hrPEDF were identified by this method (see FIGS. 6 and 7, and Table 1). The N terminal domain of hrPEDF contains L A A A * V S N F (SEQ ID NO: 1), wherein MMP-9 cleaves between the A and V, as indicated by the asterisk. The Reactive Center Loop of hrPEDF contains Q P A H * L T F P (SEQ ID NO: 2), wherein MMP-9 cleaves between the H and L, as indicated by the asterisk.

There are at least 34 other potential cleavage sites on PEDF as determined by the consensus sequence for MMP-9 (see FIG. 7 and Table 1). The consensus sequence of MMP-9 is X P R S/T * Hy S/T X X (SEQ ID NO: 3), wherein X denotes any amino acid, Hy denotes a hydrophobic amino acid (e.g., A, C, I, L, M, F, P, W, Y, or V), and cleavage occurs between S/T and Hy as indicated by the asterisk (see FIG. 7). The consensus sequence for MMP-9 is not exact, so an additional “consensus sequence” is X Xₐ X_{b} X_{c} * Hy X_{d} Xₑ X (SEQ ID NO: 4), wherein X denotes any amino acid; Xₐ can be A, G, or L; X_{b} can be R, L, or A; X_{c} can be E, K, H, A, or R, X_{d} can be L, K, G, or A, Xₑ can be G, D, A, or N; and Hy denotes a hydrophobic amino acid as described above (see FIG. 7). Even this additional “consensus sequence” does not encompass all of the potential cleavage sites described in Table 1.

TABLE 1

| | | | | | | | | | |
|---|---|---|---|---|---|---|---|---|---|
| | | | | | | | | | SEQ |
| | | | | | | | | | ID |
| P4 | P3 | P2 | P1 | ↓ | P1′ | P2′ | P3′ | P4′ | NO: |

| | | | | | | | | | |
|---|---|---|---|---|---|---|---|---|---|
| Consensus = | X | P | R | S/T | Hy | S/T | X | X | 3 |
| | | A | L | E | | L | G | | |
| | | G | A | K | | K | A | | |
| | | L | | A | | G | D | | |
| | | | | H | | A | N | | |
| | | | | R | | | | | |
| Hy = Hydrophobic | | | | | | | | | |

| | amino acid | | | | | | | | | | ID? | Domain |
|---|---|---|---|---|---|---|---|---|---|---|---|---|
| 1 | 75-82 | G | A | E | Q | R | T | E | S | 5 | | Neurotrophic |
| 2 | 34-41 | L | A | A | A | V | S | N | F | 1 | Yes | |
| 3 | 96-103 | P | D | I | H | G | T | Y | K | 6 | | Neurotrophic |
| 4 | 201-208 | F | D | S | R | K | T | S | L | 7 | | |
| 5 | 235-242 | L | D | S | D | L | S | C | K | 8 | | |
| 6 | 213-220 | L | D | E | E | R | T | V | R | 9 | | |
| 7 | 103-110 | K | E | L | L | D | T | V | T | 10 | | |
| 8 | 303-310 | G | E | V | T | K | S | L | Q | 11 | | |
| 9 | 269-276 | I | E | E | S | L | T | S | E | 12 | | |
| 10 | 270-277 | E | E | S | L | T | S | E | F | 13 | | |
| 11 | 349-356 | A | G | T | T | P | S | P | G | 14 | | Reactive Center Loop |
| 12 | 194-201 | K | G | Q | W | V | T | K | F | 15 | | |
| 13 | 139-146 | E | K | S | Y | G | T | R | P | 16 | | |
| 14 | 295-302 | P | K | L | K | L | S | Y | E | 17 | | |
| 15 | 170-177 | G | K | L | A | R | S | T | K | 18 | | |
| 16 | 267-274 | T | L | I | E | E | S | L | T | 19 | | |
| 17 | 127-134 | K | L | R | I | K | S | S | F | 20 | | |
| 18 | 171-178 | K | L | A | R | S | T | K | E | 21 | | |
| 19 | 105-112 | L | L | D | T | V | T | A | P | 22 | | |
| 20 | 246-253 | L | P | L | T | G | S | M | S | 23 | | |
| 21 | 95-102 | S | P | D | I | H | G | T | Y | 24 | | |
| 22 | 179-186 | I | P | D | E | I | S | I | L | 25 | | |
| 23 | 136-143 | A | P | L | E | K | S | Y | G | 26 | | |
| 24 | 358-365 | Q | P | A | H | L | T | F | P | 2 | Yes | Reactive Center Loop |
| 25 | 145-152 | R | P | R | V | L | T | G | N | 27 | | |
| 26 | 329-336 | K | P | I | K | L | T | Q | V | 28 | | |
| 27 | 320-327 | S | P | D | F | S | K | I | T | 29 | | |
| 28 | 61-68 | S | P | L | S | V | A | T | A | 30 | | |
| 29 | 226-233 | D | P | K | A | V | L | R | Y | 31 | | |
| 30 | 258-265 | L | P | L | K | V | T | Q | N | 32 | | |
| 31 | 77-84 | E | Q | R | T | E | S | I | I | 33 | | |
| 32 | 315-322 | Q | S | L | F | D | S | P | D | 34 | | |
| 33 | 202-209 | D | S | R | K | T | S | L | E | 35 | | |
| 34 | 198-205 | V | T | K | F | D | S | R | K | 36 | | |
| 35 | 89-96 | Y | Y | D | L | I | S | S | P | 37 | | Neurotrophic |
| 36 | 45-52 | L | Y | R | V | R | S | S | M | 38 | | |

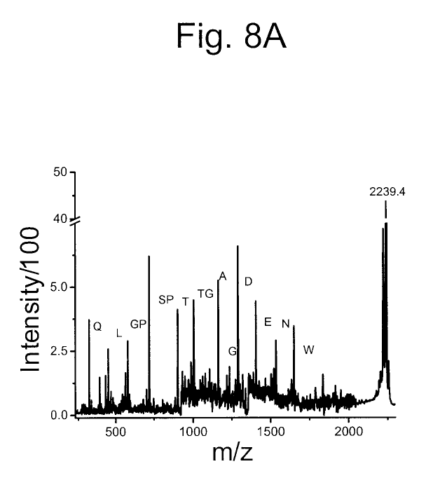

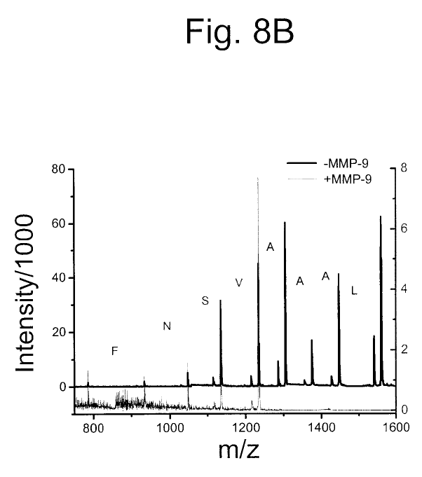

Comparison of the HPLC chromatographs indicated a few peaks that were different between the MMP-treated and untreated PEDF. Fractions corresponding to these differentially eluted peaks were selected, dried, and treated with 2-sulfobenzoic acid anhydride as described by Keough et al., Proc. Natl. Acad. Sci. USA, 96(13): 7131-36 (1999), to enhance sequencing simplicity and sensitivity. Immediately after 2-sulfobenzoic acid anhydride treatment, the peptide sequences were determined by MALDI mass spectrometric analysis on α-CHCA matrix. Two peptides were identified in the selected fractions of the MMP-treated sample whose sequence did not match that of a control tryptic digest, i.e., the observed proteolytic cleavage was not consistent with the known specificity of trypsin, and therefore, was a result of MMP-9 proteolytic cleavage (see FIGS. 8A and 8B).

The identification of MMP cleavage sites within a serpin protein (e.g., PEDF) identifies regions of the serpin protein to mutate to prevent MMP-mediated degradation to extend the duration of the serpin (e.g., in treatment of angiogenesis-related disease) in a given tissue.

### EXAMPLE 3

This example demonstrates the effect of the administration of an MMP inhibitor and a serpin protein in vivo.

Replication-deficient adenoviral vectors comprising the coding sequence for PEDF operably linked to the CMV immediate early promoter were constructed using standard techniques. E1-/E3-/E4-deficient vectors encoding PEDF with a spacer in the E4 region (AdPEDF.11D) were constructed, as described, for example, in U.S. Patent Application Publication 2003/0045498.

SB-3CT (Cal Biochem) is potent, selective, slow-binding, and mechanism-based inhibitor of human gelatinases, MMP-2 and MMP-9, which behaves similarly to TIMP-1 and TIMP-2 in the slow-binding component of inhibition.

Adult female C57BL/6 mice were intravitreally injected with approximately 1×10⁹ pu/μL of AdPEDF.11D and 1 μg/mL of MMP inhibitor, SB-3CT, either simultaneously or separately. Controls included naïve mice and mice that were intravitreally injected with AdPEDF.11D only.

For the co-treatment of AdPEDF.11D and SB-3CT, as well as the control mice that were administered only AdPEDF.11D, mice were intravitreally injected on Day 0, as described above. For the separate treatment of AdPEDF.11D and SB-3CT, mice were pre-treated with SB-3CT at Day −1 and treated with AdPEDF.11D at Day 0.

SB-3CT and/or Ad.PEDF.11D was administered to the eye via intravitreal injection. Injections were performed by forming an entrance site in the posterior portion of the eye and administering SB-3CT and/or Ad.PEDF.11D. The mice were sacrificed at Day 1, Day 3, Day 7, and Day 14. The eyes of each animal were enucleated and prepared for PEDF expression analysis by techniques known in the art (see, for example, Sambrook et al., supra).

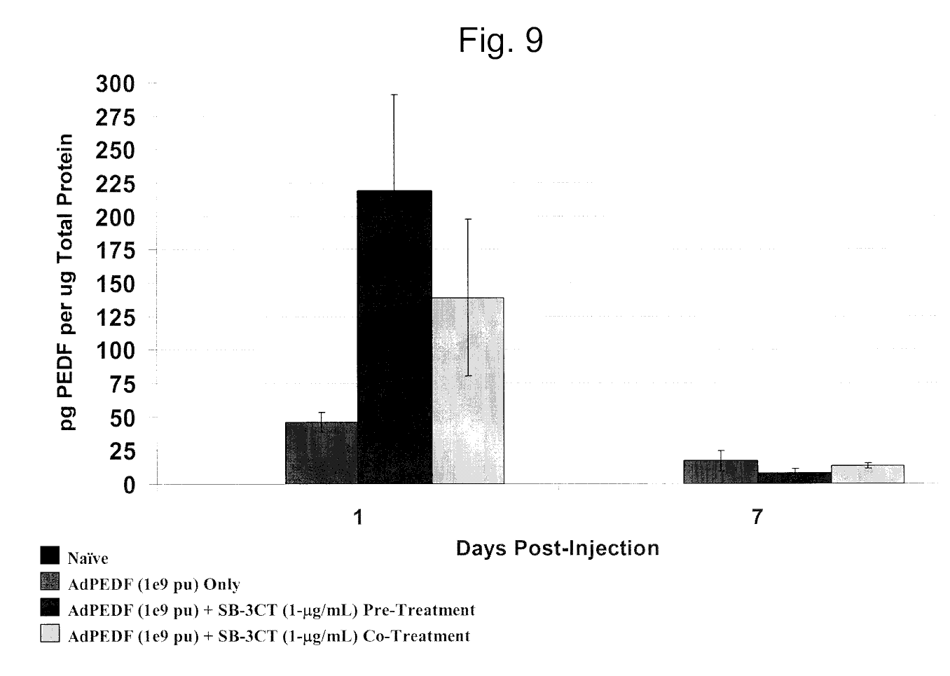

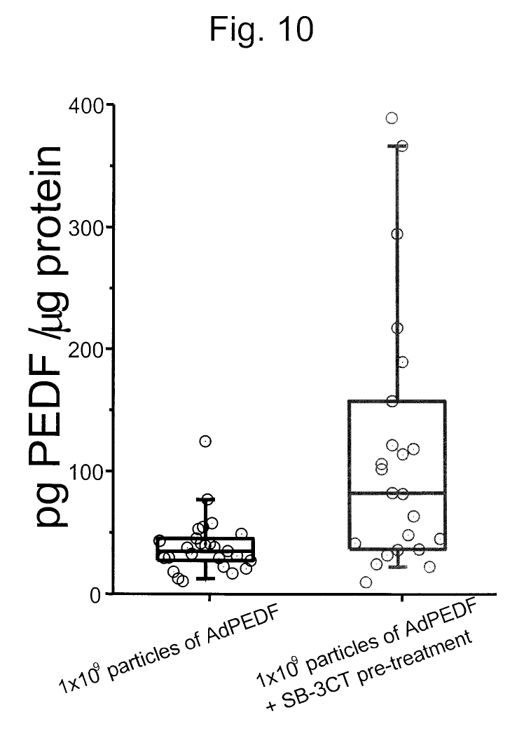

FIG. 9 illustrates the results of the analysis on Day 1 and Day 7 (post-injection). Samples from naïve animals (control) did not exhibit quantifiable levels of PEDF protein on Day 1 or Day 7. The samples from mice only administered AdPEDF.11D contained about 48 pg of PEDF protein per total μg of protein. In contrast, samples from mice co-administered AdPEDF.11D and SB-3CT (on Day 0) contained about 140 pg PEDF per total μg of protein (about 3 times as much as mice administered AdPEDF.11D only) (see FIG. 10). Interestingly, samples from mice pre-treated with SB-3CT (on Day 1) before administration of AdPEDF.11D (on Day 0) contained about 220 pg PEDF per total μg of protein (about 4.5 times as much as mice administered AdPEDF.11D only).

However, by Day 7, the samples from the mice administered SB-3CT and/or AdPEDF.11D (co-treatment or pre-treatment) showed no significant difference in the amount of PEDF protein per total protein isolated. In the mice administered SB-3CT, this result may be due to rapid clearance of the SB-3CT from the mice, which may result in PEDF cleavage by MMPs (such as observed in mice administered AdPEDF.11D only). Experiments wherein the mice are constantly administered MMP inhibitors, such as SB-3CT, in the water supply may provide a steady dose of the MMP inhibitor, which could result in longer time span of PEDF expression and activity.

This example demonstrates that administration an MMP inhibitor results in increased expression of a serpin protein (i.e., increased stability of the protein).

### EXAMPLE 4

This example demonstrates the effect of an MMP on the activity of a serpin protein.

The effect of MMP-9-processed PEDF on inhibition of endothelial cell invasion was investigated. The invasion assay was performed according to the manufacturer instructions (BD Biosciences, San Jose, Calif.). Briefly, human dermal microvascular endothelial cells (ATCC, Manassas, Va.) were plated on tissue culture treated, T25 flasks and expanded by passaging three times. Cells were then cultured on BD Matrigel™ Matrix in the invasion assay transwells in minimal media and suspended over test media containing various concentrations of the factors VEGF plus FGF (0 or 25 and 2 ng/ml, respectively) and PEDF (from 0 to 100 ng/ml). Invading cells were quantified after staining with calcein (Molecular Probes, Eugene, Oreg.) on a Typhoon™ (Amersham Biosciences, Piscataway, N.J.) fluorescent imager.

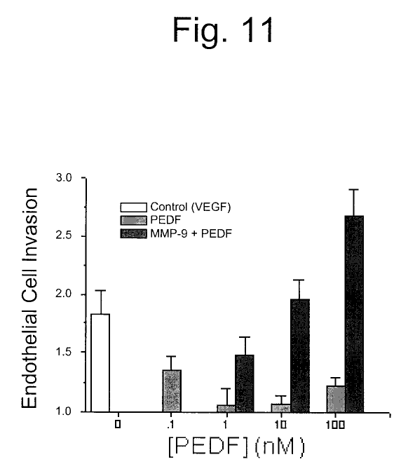

In contrast the environment of the aortic ring assay described in Example 1, the cells in the invasion assay were exposed to a gradient of tissue factors such as VEGF and PEDF, similar to the process of neovascularization where endothelial cells proliferate from established vessels in response to chemoattractive and proliferative agents and migrate into position to form a tube. Untreated PEDF potently inhibited pro-angiogenic activities; however, MMP-9-treated PEDF not only was unable to inhibit endothelial cell invasion, but also acted as a chemoattractant for endothelial cells (see FIG. 11).

The results of this Example suggest a previously unknown control mechanism for angiogenesis by the conversion of PEDF from anti-angiogenic to pro-angiogenic activity in certain pathological conditions.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inhibiting angiogenesis within a tissue, which method comprises contacting the tissue with at least one of the following:
(i) a composition comprising (a) a gene transfer vector comprising a nucleic acid sequence encoding a protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed;
(ii) a gene transfer vector comprising a nucleic acid sequence encoding a protein of a serpin superfamily or a therapeutic fragment or variant thereof, wherein the nucleic acid sequence comprises at least one mutation which renders the protein of the serpin superfamily resistant to cleavage by a MMP, and wherein the nucleic acid sequence encoding the protein of the serpin superfamily or a therapeutic fragment or variant thereof is expressed;
(iii) a composition comprising (a) a protein of a serpin superfamily or a therapeutic fragment or variant thereof and (b) an inhibitor of a MMP; and
(iv) a protein of a serpin superfamily or a therapeutic fragment or variant thereof, wherein the protein of the serpin superfamily is resistant to cleavage by a MMP;
wherein angiogenesis within the tissue is inhibited.

2. The method of claim 1, wherein the at least one mutation is in the nucleic acid sequence encoding SEQ ID NO: 1, SEQ ID NO: 2, SEQ ID NO: 3, or SEQ ID NO: 4.

3. The method of claim 1, wherein the inhibitor of an MMP is tissue inhibitor of metalloproteinases (TIMP) or a therapeutic fragment or variant thereof.

4. The method of claim 3, wherein the TIMP is selected from the group consisting of TIMP-1, TIMP-2, TIMP-3, and TIMP-4.

5. The method of claim 1, wherein the protein of a serpin superfamily or variant thereof is pigment epithelium-derived factor (PEDF) or a therapeutic fragment or variant thereof.

6. The method of claim 1, wherein the MMP is selected from the group consisting of MMP-1, MMP-3, MMP-7, MMP-9, and MMP-12.

7. A method of promoting neuron protection within a tissue, which method comprises contacting the tissue with at least one of the following:
(i) a composition comprising (a) a gene transfer vector comprising a nucleic acid sequence encoding a protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed;
(ii) a gene transfer vector comprising a nucleic acid sequence encoding a protein of a serpin superfamily or a therapeutic fragment or variant thereof, wherein the nucleic acid sequence comprises at least one mutation which renders the protein of the serpin superfamily resistant to cleavage by a MMP, and wherein the nucleic acid sequence encoding the protein of the serpin superfamily or a therapeutic fragment or variant thereof is expressed;
(iii) a composition comprising (a) a protein of a serpin superfamily or a therapeutic fragment or variant thereof and (b) an inhibitor of a MMP; and
(iv) a protein of a serpin superfamily or a therapeutic fragment or variant thereof, wherein the protein of the serpin superfamily is resistant to cleavage by a MMP;
wherein neuron protection within the tissue is promoted.

8. The method of claim 7, wherein the at least one mutation is in the nucleic acid sequence encoding SEQ ID NO: 1, SEQ ID NO: 2, SEQ ID NO: 3, or SEQ ID NO: 4.

9. The method of claim 7, wherein the inhibitor of an MMP is tissue inhibitor of metalloproteinases (TIMP) or a therapeutic fragment or variant thereof.

10. The method of claim 9, wherein the TIMP is selected from the group consisting of TIMP-1, TIMP-2, TIMP-3, and TIMP-4.

11. The method of claim 7, wherein the protein of a serpin superfamily or variant thereof is pigment epithelium-derived factor (PEDF) or a therapeutic fragment or variant thereof.

12. The method of claim 7, wherein the MMP is selected from the group consisting of MMP-1, MMP-3, MMP-7, MMP-9, and MMP-12.

13. A method of treating a disease in a mammal, which method comprises administering to the mammal at least one of the following:
(i) a composition comprising (a) a gene transfer vector comprising a nucleic acid sequence encoding a protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed;
(ii) a gene transfer vector comprising a nucleic acid sequence encoding a protein of a serpin superfamily or a therapeutic fragment or variant thereof, wherein the nucleic acid sequence comprises at least one mutation which renders the protein of the serpin superfamily resistant to cleavage by a MMP, and wherein the nucleic acid sequence encoding the protein of the serpin superfamily or a therapeutic fragment or variant thereof is expressed;
(iii) a composition comprising (a) a protein of a serpin superfamily or a therapeutic fragment or variant thereof and (b) an inhibitor of a MMP; and
(iv) a protein of a serpin superfamily or a therapeutic fragment or variant thereof, wherein the protein of the serpin superfamily is resistant to cleavage by a MMP;
thereby treating the disease in the mammal.

14. The method of claim 13, wherein the at least one mutation is in the nucleic acid sequence encoding SEQ ID NO: 1, SEQ ID NO: 2, SEQ ID NO: 3, or SEQ ID NO: 4.

15. The method of claim 13, wherein the inhibitor of an MMP is tissue inhibitor of metalloproteinases (TIMP) or a therapeutic fragment or variant thereof.

16. The method of claim 15, wherein the TIMP is selected from the group consisting of TIMP-1, TIMP-2, TIMP-3, and TIMP-4.

17. The method of claim 13, wherein the protein of a serpin superfamily or variant thereof is pigment epithelium-derived factor (PEDF) or a therapeutic fragment or variant thereof.

18. The method of claim 13, wherein the MMP is selected from the group consisting of MMP-1, MMP-3, MMP-7, MMP-9, and MMP-12.

19. A method of inhibiting vascular permeability in a tissue, which method comprises administering to the mammal at least one of the following:
(i) a composition comprising (a) a gene transfer vector comprising a nucleic acid sequence encoding protein of a serpin superfamily or a therapeutic fragment or variant thereof and (b) an inhibitor of a matrix metalloprotease (MMP), wherein the nucleic acid sequence encoding the protein of a serpin superfamily or a therapeutic fragment or variant thereof is expressed; and
(ii) a composition comprising (a) a protein of a serpin superfamily or a therapeutic fragment or variant thereof and (b) an inhibitor of a MMP;
thereby inhibiting vascular permeability in the tissue.

20. The method of claim 9, wherein the inhibitor of an MMP is tissue inhibitor of metalloproteinases (TIMP) or a therapeutic fragment or variant thereof.
